(19) United States
(12) Patent Application Publication
(10) Pub. No.: US 2014/0206629 A1
(43) Pub. Date: Jul. 24, 2014

(54) Stromal Derived Factor Inhibition And CXCR4 Blockade

(71) Applicant: Theodore A. Blaine, Old Greenwich, CT (US)

(72) Inventor: Theodore A. Blaine, Old Greenwich, CT (US)

(73) Assignee: RHODE ISLAND HOSPITAL, Providence, RI (US)

(21) Appl. No.: 13/985,415

(22) PCT Filed: Feb. 17, 2012
(86) PCT No.: PCT/US12/25686

§ 371 Date: Mar. 27, 2014

Related U.S. Application Data
(60) Provisional application No. 61/444,011, filed on Feb. 17, 2011.

(51) Int. Cl. C07D 295/135; C07C 211/27; A61K 38/10; C07C 237/30; C07D 233/88; C07C 257/18; C07D 233/64

(52) U.S. Cl. CPC C07D 295/135; C07C 257/18; C07C 211/27; C07D 233/64; C07C 237/30; C07D 233/88; A61K 38/10
USPC 514/21.5; 514/636; 514/637; 514/655; 514/397; 514/231.8; 514/616; 514/634; 514/402

(57) ABSTRACT

The invention relates to inhibition of SDF-1α expression in subacromial bursa cells by CXCR-4 inhibitors. Bursal cell migration in response to SDF-α stimulation is also decreased in the presence of CXCR4 inhibitors. Accordingly, provided are methods for treating or ameliorating a musculoskeletal disorder.

### RELATED APPLICATIONS

This application claims priority to, and the benefit of, U.S. Patent Application No. 61/444,011, filed Feb. 17, 2011, which is incorporated herein by reference in its entirety.

### FIELD OF THE INVENTION

This present invention generally relates to methods for treating a musculoskeletal disorder.

### BACKGROUND OF THE INVENTION

Musculoskeletal disorders are a large group of common diseases and disorders which can involve bone, muscles, ligaments, tendons, cartilage and joints. Musculoskeletal disorders occur frequently and include non-specific complaints such as back pain, neck pain or repetitive strain injury, chronic inflammatory or degenerative rheumatic disorders as well as a wide range of other sports injuries and work-related injuries. Bursitis and tendonitis are some of the most common musculoskeletal complaints. (Hedrick et al. U.S. Pat. No. 7,771,716). The etiology is likely multifactorial and includes both mechanical and biologic factors. It has been shown that bursitis is characterized by chronic inflammation.

Injection of corticosteroid medication is a common outpatient procedure for the treatment of bursitis, tendonitis and other musculoskeletal inflammatory conditions. However, both corticosteroids and non-steroidal anti-inflammatory drugs (NSAIDs) have systemic risks, such as induction of peptic ulcer disease and alterations in blood sugar levels, and may inhibit or impair rotator cuff tendon healing. Therefore, there is need to identify new pharmacologic agents to treat musculoskeletal disorders with fewer side effects than steroids and NSAIDs.

### SUMMARY OF THE INVENTION

This invention relates to the surprising discovery that administration of a compound that inhibits or reduces binding of Stromal Cell-Derived Factor-1α (SDF-1α) and CXCR4 reduces inflammation associated with musculoskeletal disorders, such as bursitis and tendonitis. For example, the compound reduces inflammation and promotes the healing of a rotator cuff tendon.

Thus, one aspect of the present invention relates to methods of treating or ameliorating a musculoskeletal disorder in a subject in need thereof by administering to said subject a compound that inhibits or reduces binding of SDF-1α and CXCR4. For example, the compound inhibits or binds to SDF-1α, therefore preventing or reducing the binding of SDF-1α and CXCR4. Alternatively, the compound inhibits or binds to CXCR4, which leads to prevention or reduction in the binding of SDF-1α and CXCR4. An exemplary CXCR4 blocker is a small molecule inhibitor, such as AMD 3100. Another example of a CXCR4 blocking agent is a peptide inhibitor, such as a T140 analog. Exemplary T140 analogs are SEQ ID NOs: 7-10.

Musculoskeletal disorders to be treated include bursitis and tendonitis, as well as rotator cuff tendonitis, epicondylitis olecranon bursitis, trochanteric bursitis, and patellar tendonitis. In some embodiments, the methods do not comprise treatment of rheumatoid arthritis or autoimmune disease. The compound is administered locally to a musculoskeletal tissue, such as by directly contacting a bursa or tendon or by injection, infusion onto the area. Other forms of local administration include topical administration, e.g. by transdermal patch or by application of a cream or gel to the skin at or near the affected area.

A “subject” in the context of the present invention is preferably a mammal. The mammal can be a human, non-human primate, mouse, rat, dog, cat, horse, or cow, but are not limited to these examples. A subject can be male or female. A subject can be one who has been previously diagnosed or identified as having musculoskeletal disorders, and optionally has already undergone, or is undergoing, a therapeutic intervention for the musculoskeletal disorders such as bursitis or tendonitis. Alternatively, a subject can also be one who has not been previously diagnosed as having musculoskeletal disorders, but who is at risk of developing such condition, e.g. due to injury, hereditary predisposition or infection. For example, a subject can be one who exhibits one or more symptoms for musculoskeletal disorders.

A “small molecule” as used herein, is meant to refer to a composition that has a molecular weight of less than about 5 kD and most preferably less than about 4 kD, e.g. less than 1 kD, more preferably less than 500 Daltons. Small molecules can be, e.g., nucleic acids, peptides, polypeptides, peptidomimetics, carbohydrates, lipids or other organic or inorganic molecules.

Inhibitory compounds are purified naturally-occurring, synthetically produced, or recombinant compounds, e.g., polypeptides, nucleic acids, small molecules, or other agents. The compositions used in the therapeutic methods described herein are purified. Purified compounds are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, polyacrylamide gel electrophoresis, or HPLC analysis.

Other features and advantages of the invention will be apparent from the following description of the preferred embodiments thereof, and from the claims. All references (including sequences identified by GENBANK™/NCBI accession numbers) cited herein are incorporated by reference.

### BRIEF DESCRIPTION OF THE DRAWINGS

FIG. 1 is a series of photographs showing immunohistochemistry of bursal specimens in patients with normal (negative control), bursitis, and rheumatoid (positive control). Specimens are stained with antihuman IL-1B, IL-6, and SDF-1α.

FIG. 2 is a photograph of human subacromial bursal cells in culture. Cells have a synoviocyte-like appearance.

FIG. 3 is a graph showing cell characterization by flow cytometry. Cells stained with PE Mouse Anti-Human CD44 (a), FITC Mouse Anti-Human CD106 (b), and unstained cells (c).

FIG. 4 is a bar graph showing a time course experiment. SDF-1α protein production in the supernatants of cultured bursal cells treated with interleukin 10 over time.

FIG. 5 is a bar graph showing a dose response experiment. SDF-1α protein in the supernatants of cultured bursal cells treated with inflammatory cytokines.

FIG. 6 is a bar graph showing cell migration assay of human subacromial bursal cells in the presence of CXCR4 inhibitors AMD3100 and T140.

FIG. 7 is a photograph showing infraspinatus tendon is transected at insertion to create a tendon defect simulating rotator cuff tear.

FIG. 8 is a photomicrograph of a coronal section of the right shoulder in a rat after rotator cuff injury. H&E stain. A=Acromion, H=Humeral Head, T=Tendon.

FIG. 9 is a bar graph showing maximum load in rat rotator cuff tendons with experimentally induced injuries with (AMD 3100) or without (PBS) treatment up to six weeks post-injury.

FIG. 10A-10C is a diagram showing structures of compounds 4-21 and 24-31, small molecule CXCR4 antagonists.

### DETAILED DESCRIPTION OF THE INVENTION

Stromal Cell-Derived Factor-1α (SDF-1α) has been found to play a role in inflammation associated with the bursa and with tendon. It is a potent pro-inflammatory chemokine that mediates the cell's response to mechanical or biochemical signals. Prior to the invention, SDF-1α and its receptor CXCR4 were known to play an important role in cell migration, embryonic development, and human immunodeficiency virus infection. For example, CXCR4 is the major HIV type 1 (HIV-1) co-receptor required for viral entry into the host cells. Inhibitors to the SDF-1α receptor CXCR4 have been designed and proposed for clinical use in the prevention and treatment of HIV infection (Yoon et al. Cancer Research, 67: 75187524 (2007)).

The present invention relates to the important role played by SDF-1α and its receptor CXCR4 in many musculoskeletal inflammatory conditions. Administration of a compound that inhibits or reduces binding of SDF-1α and CXCR4 reduces the musculoskeletal inflammation without the deleterious side effects of steroids and NSAIDs and promotes the healing of tendons. In contrast, previous methods based on non-selective agents (NSAIDs, corticosteroids or cortisone injections) inhibit the healing of tendons and lead to significant local (impaired rotator cuff tendon healing) and systemic (cardiovascular and gastrointestinal) adverse events. Therefore, the invention relates to methods of treating or ameliorating a musculoskeletal disorder in a subject in need thereof by administering to said subject a compound that inhibits or reduces binding of SDF-1α and CXCR4.

### Stromal Cell-Derived Factor-1α (SPF-1α)

Stromal Cell-Derived Factor-1α (SDF-1α, also called CXCL12) is a potent CXC chemokine that is produced by bone marrow and stromal cells in other tissues. SDF-1 is important in hematopoietic stem cell homing to the bone marrow and in hematopoietic stem cell quiescence.

SDF-1α nucleotide sequence (GenBank Accession No.: NM_{—}199168.3; GI No.: 291045298) is shown below. Underlined nucleotides indicate the coding sequences (CDS).

| (SEQ ID NO: 1) | |
|---|---|
| 1 | gccgcacttt cactctccgt cagccgcatt gcccgctcgg cgtccggccc ccgacccgcg |
| 61 | ctcgtccgcc cgcccgcccg cccgcccgcg ccatgaacgc caaggtcgtg gtcgtgctgg |
| 121 | tcctcgtgct gaccgcgctc tgcctcagcg acgggaagcc cgtcagcctg agctacagat |
| 181 | gcccatgccg attcttcgaa agccatgttg ccagagccaa cgtcaagcat ctcaaaattc |
| 241 | tcaacactcc aaactgtgcc cttcagattg tagcccggct gaagaacaac aacagacaag |
| 301 | tgtgcattga cccgaagcta aagtggattc aggagtacct ggagaaagct ttaaacaagt |
| 361 | aagcacaaca gccaaaaagg actttccgct agacccactc gaggaaaact aaaaccttgt |
| 421 | gagagatgaa agggcaaaga cgtgggggag ggggccttaa ccatgaggac caggtgtgtg |
| 481 | tgtggggtgg gcacattgat ctgggatcgg gcctgaggtt tgccagcatt tagaccctgc |
| 541 | atttatagca tacggtatga tattgcagct tatattcatc catgccctgt acctgtgcac |
| 601 | gttggaactt ttattactgg ggtttttcta agaaagaaat tgtattatca acagcatttt |
| 661 | caagcagtta gttccttcat gatcatcaca atcatcatca ttctcattct cattttttaa |
| 721 | atcaacgagt acttcaagat ctgaatttgg cttgtttgga gcatctcctc tgctcccctg |
| 781 | gggagtctgg gcacagtcag gtggtggctt aacagggagc tggaaaaagt gtcctttctt |
| 841 | cagacactga ggctcccgca gcagcgcccc tcccaagagg aaggcctctg tggcactcag |
| 901 | ataccgactg gggctgggcg ccgccactgc cttcacctcc tctttcaacc tcagtgattg |
| 961 | gctctgtggg ctccatgtag aagccactat tactgggact gtgctcagag acccctctcc |
| 1021 | cagctattcc tactctctcc ccgactccga gagcatgctt aatcttgctt ctgcttctca |
| 1081 | tttctgtagc ctgatcagcg ccgcaccagc cgggaagagg gtgattgctg gggctcgtgc |
| 1141 | cctgcatccc tctcctccca gggcctgccc cacagctcgg gccctctgtg agatccgtct |
| 1201 | ttggcctcct ccagaatgga gctggccctc tcctggggat gtgtaatggt ccccctgctt |
| 1261 | acccgcaaaa gacaagtctt tacagaatca aatgcaattt taaatctgag agctcgcttt |
| 1321 | gagtgactgg gttttgtgat tgcctctgaa gcctatgtat gccatggagg cactaacaaa |
| 1381 | ctctgaggtt tccgaaatca gaagcgaaaa aatcagtgaa taaaccatca tcttgccact |
| 1441 | accccctcct gaagccacag cagggtttca ggttccaatc agaactgttg gcaaggtgac |
| 1501 | atttccatgc ataaatgcga tccacagaag gtcctggtgg tatttgtaac tttttgcaag |
| 1561 | gcattttttt atatatattt ttgtgcacat ttttttttac gtttctttag aaaacaaatg |
| 1621 | tatttcaaaa tatatttata gtcgaacaat tcatatattt gaagtggagc catatgaatg |
| 1681 | tcagtagttt atacttctct attatctcaa actactggca atttgtaaag aaatatatat |
| 1741 | gatatataaa tgtgattgca gcttttcaat gttagccaca gtgtattttt tcacttgtac |
| 1801 | taaaattgta tcaaatgtga cattatatgc actagcaata aaatgctaat tgtttcatgg |
| 1861 | tataaacgtc ctactgtatg tgggaattta tttacctgaa ataaaattca ttagttgtta |
| 1921 | gtgatggagc ttaaaaaaaa |

SDF-1α protein sequence (GenBank Accession No.: NP_{—}954637.1; GI No.: 40316924) is:

(SEQ ID NO: 2)
mnakvvvvlv lvltalclsd gkpvslsyrc pcrffeshva ranvkhlkil ntpncalqiv arlknnnrqv
cidpklkwiq eylekalnk.

Underlined residues indicate the sequence of the mature protein.

### CXCR4

CXCR4 is an α-chemokine receptor specific for SDF-1, a molecule endowed with potent chemotactic activity for lymphocytes. This receptor is one of several chemokine receptors that HIV isolates can use to infect CD4+ T cells. CXCR4 is upregulated during the implantation window in natural and hormone replacement therapy cycles in the endometrium, producing, in presence of a human blastocyst, a surface polarization of the CXCR4 receptors suggesting that this receptor is implicated in the adhesion phase of human implantation.

The binding of chemokine receptors to their natural ligands appears to serve a more evolutionary and central role than only as mediators of HIV infection. The binding of SDF-1 to the CXCR4 chemokine receptor provides an important signaling mechanism: CXCR4 or SDF-1 knock-out mice exhibit cerebellar, haematopoiesis development and gastrointestinal tract abnormalities and die in utero (Zou et al., Nature, 393:595-599 (1998); Tachibana et al., Nature, 393:591-594 (1998)). CXCR4-deficient mice also display hematopoietic defects (Nagasawa et al. Nature 382, 635-638 (1996)). The signal provided by SDF-1 on binding to CXCR4 may also play an important role in tumor cell proliferation and regulation of angiogenesis associated with tumor growth (Seghal et al. J. Surg. Oncol. 69, 99-104 (1998)). The binding of SDF-1 to CXCR4 has also been implicated in the pathogenesis of atherosclerosis (Abi-Younes et al. Circ. Res. 86, 131-138 (2000)), renal allograft rejection (Eitner et al. Transplantation 66, 1551-1557 (1998)), asthma and allergic airway inflammation (Yssel et al. Clinical and Experimental Allergy 28(s5), 104-109 (1998); Gonzalo et al. J. Immunol. 165, 499-508 (2000)), Alzheimer's disease (Xia et al. J. Neurovirology 5, 32-41 (1999)) and Arthritis (Nanki et al. J. Immunol. 164, 5010-5014 (2000)).

CXCR4 has two transcript variants. The variant 2 contains a distinct 5′ UTR and lacks an in-frame portion of the 5′ coding region, compared to the variant 1. The resulting isoform b has a shorter N-terminus when compared to isoform a.

The nucleotide of CXCR4 variant 1 (GenBank Accession No.: NM_{—}001008540.1; GI No.: 56790926) is:

| (SEQ ID NO: 3) | |
|---|---|
| 1 | ttttttttct tccctctagt gggcggggca gaggagttag ccaagatgtg actttgaaac |
| 61 | cctcagcgtc tcagtgccct tttgttctaa acaaagaatt ttgtaattgg ttctaccaaa |
| 121 | gaaggatata atgaagtcac tatgggaaaa gatggggagg agagttgtag gattctacat |
| 181 | taattctctt gtgcccttag cccactactt cagaatttcc tgaagaaagc aagcctgaat |
| 241 | tggtttttta aattgcttta aaaatttttt ttaactgggt taatgcttgc tgaattggaa |
| 301 | gtgaatgtcc attcctttgc ctcttttgca gatatacact tcagataact acaccgagga |
| 361 | aatgggctca ggggactatg actccatgaa ggaaccctgt ttccgtgaag aaaatgctaa |
| 421 | tttcaataaa atcttcctgc ccaccatcta ctccatcatc ttcttaactg gcattgtggg |
| 481 | caatggattg gtcatcctgg tcatgggtta ccagaagaaa ctgagaagca tgacggacaa |
| 541 | gtacaggctg cacctgtcag tggccgacct cctctttgtc atcacgcttc ccttctgggc |
| 601 | agttgatgcc gtggcaaact ggtactttgg gaacttccta tgcaaggcag tccatgtcat |
| 661 | ctacacagtc aacctctaca gcagtgtcct catcctggcc ttcatcagtc tggaccgcta |
| 721 | cctggccatc gtccacgcca ccaacagtca gaggccaagg aagctgttgg ctgaaaaggt |
| 781 | ggtctatgtt ggcgtctgga tccctgccct cctgctgact attcccgact tcatctttgc |
| 841 | caacgtcagt gaggcagatg acagatatat ctgtgaccgc ttctacccca atgacttgtg |
| 901 | ggtggttgtg ttccagtttc agcacatcat ggttggcctt atcctgcctg gtattgtcat |
| 961 | cctgtcctgc tattgcatta tcatctccaa gctgtcacac tccaagggcc accagaagcg |
| 1021 | caaggccctc aagaccacag tcatcctcat cctggctttc ttcgcctgtt ggctgcctta |
| 1081 | ctacattggg atcagcatcg actccttcat cctcctggaa atcatcaagc aagggtgtga |
| 1141 | gtttgagaac actgtgcaca agtggatttc catcaccgag gccctagctt tcttccactg |
| 1201 | ttgtctgaac cccatcctct atgctttcct tggagccaaa tttaaaacct ctgcccagca |
| 1261 | cgcactcacc tctgtgagca gagggtccag cctcaagatc ctctccaaag gaaagcgagg |
| 1321 | tggacattca tctgtttcca ctgagtctga gtcttcaagt tttcactcca gctaacacag |
| 1381 | atgtaaaaga ctttttttta tacgataaat aacttttttt taagttacac atttttcaga |
| 1441 | tataaaagac tgaccaatat tgtacagttt ttattgcttg ttggattttt gtcttgtgtt |
| 1501 | tctttagttt ttgtgaagtt taattgactt atttatataa attttttttg tttcatattg |
| 1561 | atgtgtgtct aggcaggacc tgtggccaag ttcttagttg ctgtatgtct cgtggtagga |
| 1621 | ctgtagaaaa gggaactgaa cattccagag cgtgtagtga atcacgtaaa gctagaaatg |
| 1681 | atccccagct gtttatgcat agataatctc tccattcccg tggaacgttt ttcctgttct |
| 1741 | taagacgtga ttttgctgta gaagatggca cttataacca aagcccaaag tggtatagaa |
| 1801 | atgctggttt ttcagttttc aggagtgggt tgatttcagc acctacagtg tacagtcttg |
| 1861 | tattaagttg ttaataaaag tacatgttaa acttaaaaaa aaaaaaaaaa aa |

Underlined nucleotides indicate the coding sequences (CDS).

The protein product of the variant 1 is the isoform a. The protein sequence of the isoform a (GenBank Accession No.: NP_{—}001008540.1; GI No.: 56790927) is:

(SEQ ID NO: 4)

| | |
|---|---|
| 1 | msiplpllqi ytsdnyteem gsgdydsmke pcfreenanf |
| | nkiflptiys iifltgivgn |
| 61 | glvilvmgyq kklrsmtdky rlhlsvadll fvitlpfwav |
| | davanwyfgn flckavhviy |
| 121 | tvnlyssvli lafisldryl aivhatnsqr prkllaekvv |
| | yvgvwipall ltipdfifan |
| 181 | vseaddryic drfypndlwv vvfqfqhimv glilpgivil |
| | scyciiiskl shskghqkrk |
| 241 | alkttvilil affacwlpyy igisidsfil leiikqgcef |
| | entvhkwisi tealaffhcc |
| 301 | lnpilyaflg akfktsaqha ltsysrgssl kilskgkrgg |
| | hssysteses ssfhss |

The nucleotide of CXCR4 variant 2 (GenBank Accession No.: NM_{—}003467.2; GI No.: 56790928) is:

| (SEQ ID NO: 5) | |
|---|---|
| 1 | aacttcagtt tgttggctgc ggcagcaggt agcaaagtga cgccgagggc ctgagtgctc |
| 61 | cagtagccac cgcatctgga gaaccagcgg ttaccatgga ggggatcagt atatacactt |
| 121 | cagataacta caccgaggaa atgggctcag gggactatga ctccatgaag gaaccctgtt |
| 181 | tccgtgaaga aaatgctaat ttcaataaaa tcttcctgcc caccatctac tccatcatct |
| 241 | tcttaactgg cattgtgggc aatggattgg tcatcctggt catgggttac cagaagaaac |
| 301 | tgagaagcat gacggacaag tacaggctgc acctgtcagt ggccgacctc ctctttgtca |
| 361 | tcacgcttcc cttctgggca gttgatgccg tggcaaactg gtactttggg aacttcctat |
| 421 | gcaaggcagt ccatgtcatc tacacagtca acctctacag cagtgtcctc atcctggcct |
| 481 | tcatcagtct ggaccgctac ctggccatcg tccacgccac caacagtcag aggccaagga |
| 541 | agctgttggc tgaaaaggtg gtctatgttg gcgtctggat ccctgccctc ctgctgacta |
| 601 | ttcccgactt catctttgcc aacgtcagtg aggcagatga cagatatatc tgtgaccgct |
| 661 | tctaccccaa tgacttgtgg gtggttgtgt tccagtttca gcacatcatg gttggcctta |
| 721 | tcctgcctgg tattgtcatc ctgtcctgct attgcattat catctccaag ctgtcacact |
| 781 | ccaagggcca ccagaagcgc aaggccctca agaccacagt catcctcatc ctggctttct |
| 841 | tcgcctgttg gctgccttac tacattggga tcagcatcga ctccttcatc ctcctggaaa |
| 901 | tcatcaagca agggtgtgag tttgagaaca ctgtgcacaa gtggatttcc atcaccgagg |
| 961 | ccctagcttt cttccactgt tgtctgaacc ccatcctcta tgctttcctt ggagccaaat |
| 1021 | ttaaaacctc tgcccagcac gcactcacct ctgtgagcag agggtccagc ctcaagatcc |
| 1081 | tctccaaagg aaagcgaggt ggacattcat ctgtttccac tgagtctgag tcttcaagtt |
| 1141 | ttcactccag ctaacacaga tgtaaaagac ttttttttat acgataaata actttttttt |
| 1201 | aagttacaca tttttcagat ataaaagact gaccaatatt gtacagtttt tattgcttgt |
| 1261 | tggatttttg tcttgtgttt ctttagtttt tgtgaagttt aattgactta tttatataaa |
| 1321 | ttttttttgt ttcatattga tgtgtgtcta ggcaggacct gtggccaagt tcttagttgc |
| 1381 | tgtatgtctc gtggtaggac tgtagaaaag ggaactgaac attccagagc gtgtagtgaa |
| 1441 | tcacgtaaag ctagaaatga tccccagctg tttatgcata gataatctct ccattcccgt |
| 1501 | ggaacgtttt tcctgttctt aagacgtgat tttgctgtag aagatggcac ttataaccaa |
| 1561 | agcccaaagt ggtatagaaa tgctggtttt tcagttttca ggagtgggtt gatttcagca |
| 1621 | cctacagtgt acagtcttgt attaagttgt taataaaagt acatgttaaa cttaaaaaaa |
| 1681 | aaaaaaaaaa a |

Underlined nucleotides indicate the coding sequences (CDS).

The protein product of the variant 2 is the isoform b. The protein sequence of the isoform b (GenBank Accession No.: NP_{—}003458.1; GI No.: 4503175) is:

| (SEQ ID NO: 6) | |
|---|---|
| 1 | megisiytsd nyteemgsgd ydsmkepcfr eenanfnkif lptiysiifl tgivgnglvi |
| 61 | lvmgyqkklr smtdkyrlhl svadllfvit lpfwavdava nwyfgnflck avhviytvnl |
| 121 | yssvlilafi sldrylaivh atnsqrprkl laekvvyvgv wipallltip dfifanvsea |
| 181 | ddryicdrfy pndlwvvvfq fqhimvglil pgivilscyc iiisklshsk ghqkrkalkt |
| 241 | tvililaffa cwlpyyigis idsfilleii kqgcefentv hkwisiteal affhcclnpi |
| 301 | lyaflgakfk tsaqhaltsv srgsslkils kgkrgghssv stesesssfh ss |

### Inhibitors of SDF/CXCR4 Binding

### Small Molecule Inhibitors

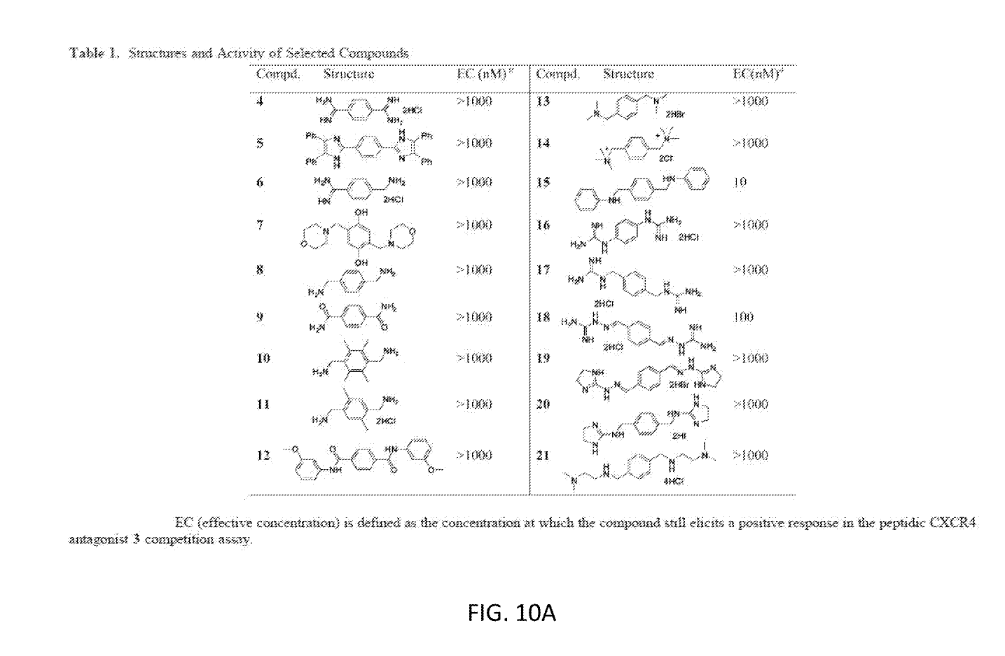

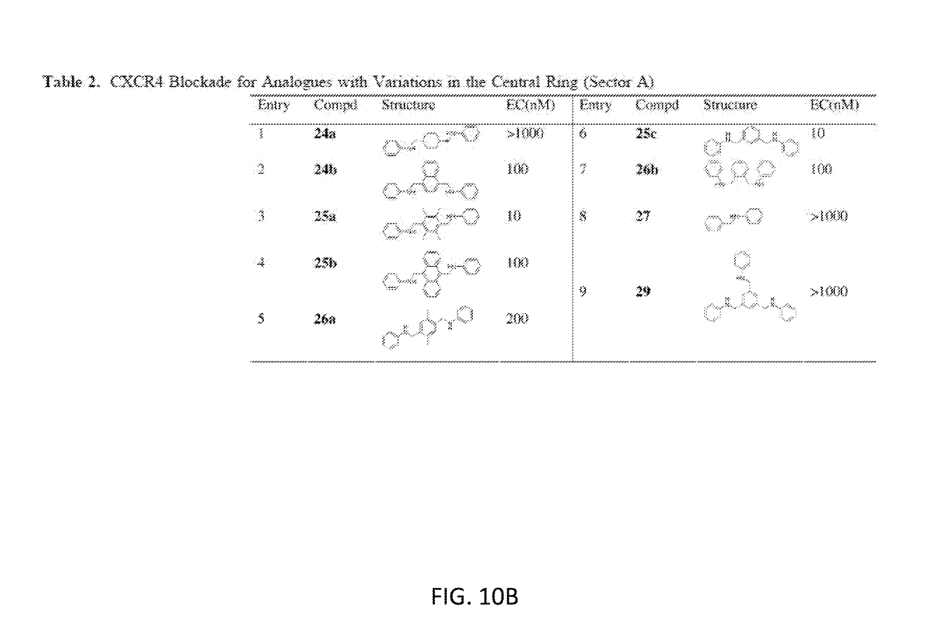

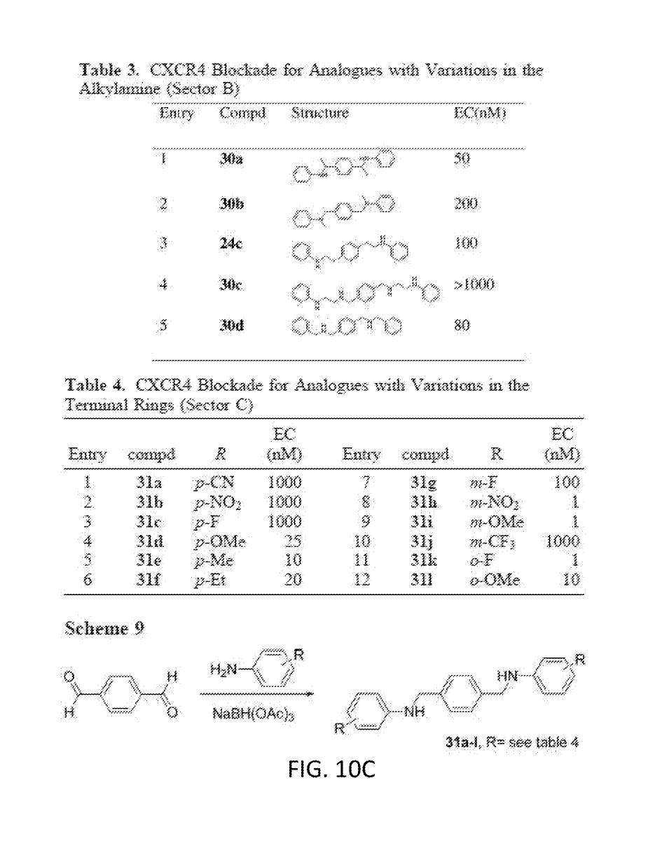

Useful small molecule inhibitors of CXCR4 receptor include without limitation AMD 3100, and compounds 4-21 and 24-32 in Table 1-4 in Zhang et al. (J. Med. Chem. 50:5655-5664 (2007)), hereby incorporated by reference (FIG. 10A-C).

AMD 3100 (1,1′-[1,4-phenylenebis(methylene)]-bis-1,4,8,11-azatetradecane) is a bicyclam compound that selectively inhibits bindings of SDF-1 to CXCR4 receptor. Previous studies showed that it inhibits the entry of human immunodeficiency virus type 1 (HIV-1) into CD4+ T cells via selective blockade of the chemokine CXCR4 receptor. (Hendrix et al. Antimicrobial Agents and Chemotherapy, 44: 1667-1673 (2000)). AMD 3100 does not bind to the other physiologically relevant chemokine receptor, CCR5.

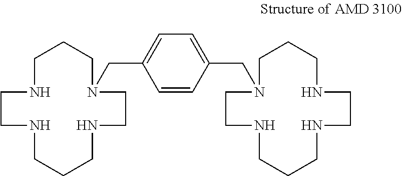 [SMILES: c1cc(CN2CCCNCCNCCCNCC2)ccc1CN1CCCNCCNCCCNCC1]

Plerixafor (Mozobil, AMD 3100) has orphan drug status for use in cancer patients to mobilize hematopoietic stem cells into peripheral blood for collection and subsequent transplantation. (Micallef et al. Bone Marrow Transplantation, Epub ahead of print (2010)). Plerixafor is currently approved for use in patients with non-hodgkins lymphoma and multiple myeloma. In some embodiments of the present invention, AMD 3100 may inhibit inflammation by reducing SDF-1α production and activity. In another embodiment of the present invention, AMD3100 causes only a minimal decrease in SDF-1α mRNA expression.

The structure of another potent compound 32 (WZ811, N,N′-Di-2-pyridinyl-1,4-benzenedimethanamine) is shown below.

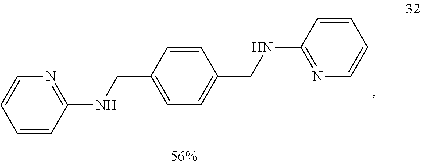 [SMILES: c1ccc(NCc2ccc(CNc3ccccn3)cc2)nc1]

In one embodiment, a small molecule inhibitor of CXCR4 receptor is a compound having the formula I:

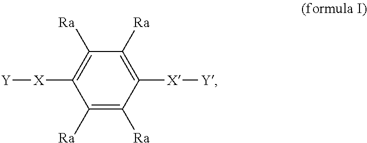

or a pharmaceutically acceptable salt, solvate, or prodrug thereof, where X and X′ are independently selected from (a) a bond, (b) C₁-C₆ alkyl, (c) C₂-C₆ alkenyl, (d) C═NR¹, (e) CO, (f) C(O)NR¹, (g) NR¹, and (h) CHNNR¹;

wherein (b)-(c) is optionally substituted with one or more —N— or —NR¹—;

Y and Y′ are independently selected from (a) NR¹R¹, (b) C(═NR¹)NR¹R¹, (c) C₃-C₁₈ membered saturated, unsaturated, or aromatic carbocycle, and (d) C₃-C₁₈ membered saturated, unsaturated, or aromatic heterocycle containing one or more heteroatoms selected from nitrogen, oxygen, or sulfur;

wherein (c)-(d) is optionally substituted with one or more R² groups;

alternatively, Y and Y′ are NR¹R¹R¹;

Ra, at each occurrence, independently, is selected from (a) hydrogen, (b) C₁-C₆ alkyl, (c) C₁-C₆ alkoxy, and (d) halogen;
R¹, at each occurrence, independently, is selected from (a) hydrogen and (b) C₁-C₆ alkyl; and
R², at each occurrence, independently, is selected from (a) C₁-C₆ alkyl, (b) OH, (c) C₁-C₆ alkoxy,
(d) C₃-C₁₄ membered saturated, unsaturated, or aromatic carbocycle, and (e) C₃-C₁₄ membered saturated, unsaturated, or aromatic heterocycle containing one or more heteroatoms selected from nitrogen, oxygen, or sulfur.
In one embodiment, a small molecule inhibitor of CXCR4 receptor is a compound having the formula Ia:

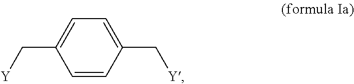

or a pharmaceutically acceptable salt, solvate, or prodrug thereof; wherein Y and Y′ are as defined above.

In one embodiment, a small molecule inhibitor of CXCR4 receptor is a compound of formula I or Ia, wherein Y and Y′ are independently a C₃-C₁₈ membered saturated, unsaturated, or aromatic heterocycle containing one or more heteroatoms selected from nitrogen, oxygen, or sulfur.

In another embodiment, a small molecule inhibitor of CXCR4 receptor is a compound of formula I or Ia, wherein Y and Y′ are independently a C₅-C₁₄ membered saturated, unsaturated, or aromatic heterocycle containing one or more heteroatoms selected from nitrogen, oxygen, or sulfur. In another embodiment, Y and Y′ are independently a C₅-C₁₄ membered saturated, unsaturated, or aromatic heterocycle containing one or more nitrogen. In another embodiment, Y and Y′ are independently a C₅-C₁₄ membered saturated, unsaturated, or aromatic heterocycle containing one or more oxygen. In another embodiment, Y and Y′ are independently a C₅-C₁₄ membered saturated, unsaturated, or aromatic heterocycle containing one or more sulfur.

In one embodiment, a small molecule inhibitor of CXCR4 receptor is a compound of formula I or Ia, wherein Y and Y′ are a C₁₄ membered saturated, unsaturated, or aromatic heterocycle containing one or more nitrogen. In another embodiment, Y and Y′ are a C₁₄ membered saturated, unsaturated, or aromatic heterocycle containing four nitrogens.

In one embodiment, a small molecule inhibitor of CXCR4 receptor is a compound of formula I or Ia, wherein Y and Y′ are NR¹R¹. In another embodiment, R¹ is hydrogen.

In one embodiment, a small molecule inhibitor of CXCR4 receptor is a compound or salt selected from:

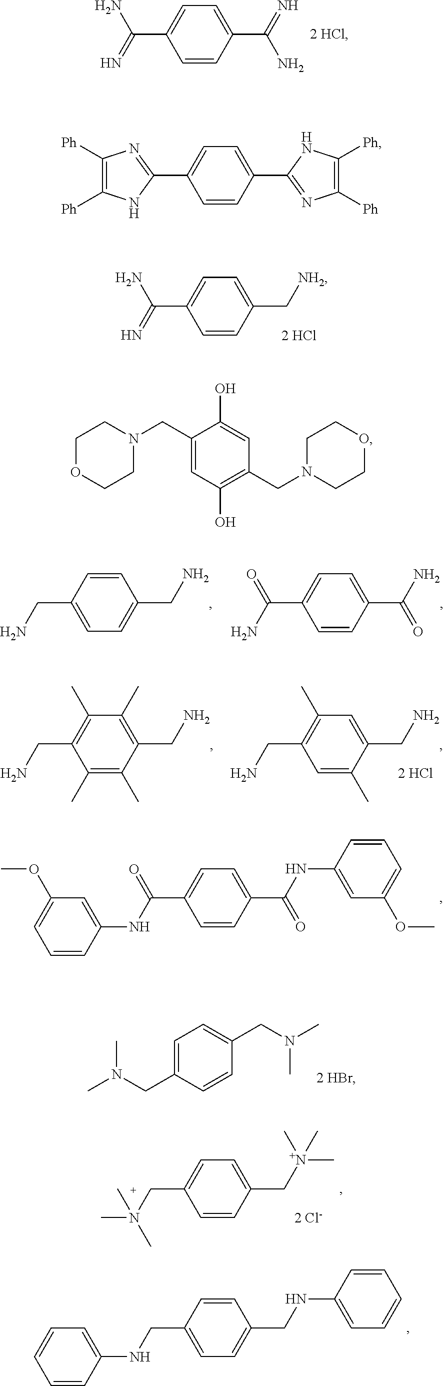

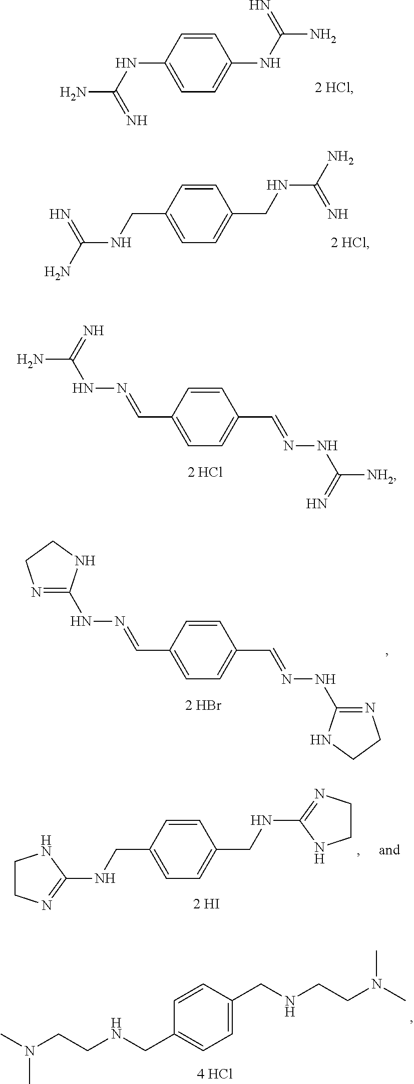

or a pharmaceutically acceptable salt, solvate, or prodrug thereof.

### DEFINITIONS

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

“Solvates” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) mifepristone or its derivative can be delivered in prodrug form. Thus, the present invention is intended to cover prodrugs of mifepristone or its derivative, methods of delivering the same and compositions containing the same. “Prodrugs” are intended to include any covalently bonded carriers that release an active parent drug of the present invention in vivo when such prodrug is administered to a subject. Prodrugs the present invention are prepared by modifying functional groups present in the compound in such a way that the modifications are cleaved, either in routine manipulation or in vivo, to the parent compound. Prodrugs include mifepristone or its derivative wherein a hydroxy, amino, sulfhydryl, carboxy, or carbonyl group is bonded to any group that, may be cleaved in vivo to form a free hydroxyl, free amino, free sulfhydryl, free carboxy or free carbonyl group, respectively.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, s-pentyl, and n-hexyl. “Alkyl” further includes alkyl groups that have oxygen, nitrogen, sulfur or phosphorous atoms replacing one or more hydrocarbon backbone carbon atoms. In certain embodiments, a straight chain or branched chain alkyl has six or fewer carbon atoms in its backbone (e.g., C₁-C₆ for straight chain, C₃-C₆ for branched chain), and more preferably four or fewer. Likewise, preferred cycloalkyls have from three to eight carbon atoms in their ring structure, and more preferably have five or six carbons in the ring structure.

The term “alkyl” also includes both “unsubstituted alkyls” and “substituted alkyls”, the latter of which refers to alkyl moieties having substituents replacing a hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl(benzyl)).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups, which include oxygen, nitrogen, sulfur or phosphorous atoms replacing one or more hydrocarbon backbone carbons. In certain embodiments, a straight chain or branched chain alkenyl group has six or fewer carbon atoms in its backbone (e.g., C₂-C₆ for straight chain, C₃-C₆ for branched chain). Likewise, cycloalkenyl groups may have from three to eight carbon atoms in their ring structure, and more preferably have five or six carbons in the ring structure. The term “C₂-C₆” includes alkenyl groups containing two to six carbon atoms. The term “C₃-C₆” includes alkenyl groups containing three to six carbon atoms.

The term “alkenyl” also includes both “unsubstituted alkenyls” and “substituted alkenyls”, the latter of which refers to alkenyl moieties having substituents replacing a hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkoxy” or “alkoxyl” includes substituted and unsubstituted alkyl, alkenyl, and alkynyl groups covalently linked to an oxygen atom. Examples of alkoxy groups (or alkoxyl radicals) include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, and trichloromethoxy.

As used herein, “halo” or “halogen” refers to fluoro, chloro, bromo, and iodo.

As used herein, “carbocycle” or “carbocyclic ring” is intended to mean any stable monocyclic, bicyclic, or tricyclic ring having the specified number of carbons, any of which may be saturated, unsaturated, or aromatic. For example a C₃₋₁₄ carbocycle is intended to mean a mono-, bi-, or tricyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, the term “heterocycle” or “heterocyclic” is intended to mean any stable monocyclic, bicyclic, or tricyclic ring which is saturated, unsaturated, or aromatic and comprises carbon atoms and one or more ring heteroatoms, e.g., 1 or 1-2 or 1-3 or 1-4 or 1-5 or 1-6 heteroatoms, independently selected from the group consisting of nitrogen, oxygen, and sulfur. A bicyclic or tricyclic heterocycle may have one or more heteroatoms located in one ring, or the heteroatoms may be located in more than one ring. The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. Bridged rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “aromatic heterocycle” or “heteroaryl” is intended to mean a stable 5, 6, or 7-membered monocyclic or bicyclic aromatic heterocyclic ring or 7, 8, 9, 10, 11, or 12-membered bicyclic aromatic heterocyclic ring which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

### CXCR4 Peptide Inhibitors

A CXCR4 peptide inhibitor is a therapeutic agent comprising an isolated and purified chemokine peptide, chemokine peptide variant, chemokine analog, or a derivative thereof.

Preferably, the therapeutic agent of the invention inhibits the activity of CXCR4. A CXCR4 peptide inhibitor is selected from the group consisting of CTCE-9908, T140, TC14012, and TC14003.

CTCE-9908 is an SDF-1 analog consisting of a dimer of the first 8 amino acids of SDF-1 and serves as a competitive inhibitor to SDF-1. CTCE-9908 competitively binds to CXCR4 and prevents bindings of SDF-1 to CXCR4 receptor. CTCE-9908 has previously been reported to inhibit metastasis in osteosarcoma, melanoma, prostate and breast cancer mouse models. (Hassan et al. International Journal of Cancer (2010); Huang et al. Journal of Surgical Research, 155: 231-236 (2009)).

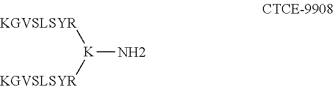

T140 and its analogs (TC14012, TE14005, and TN14003) are peptidic CXCR4 antagonists composed of 14 amino acid residues. It binds to extracellular domain of CXCR4 and inhibits bindings of SDF-1 to CXCR4 receptor. (Burger et al. Blood, 106: 1824-1830 (2005)). The present invention shows that T140 analog was the more potent inhibitor, decreasing SDF-1α protein production by 60%. The present invention further demonstrates that T140 analog significantly decreased SDF-1α mRNA expression.

(SEQ ID NO: 7)
T140 H-Arg-Arg-Nal-Cys-Tyr-Arg-Lys-_{D}-Lys-Pro-Tyr-Arg-Cit-Cys-Arg-OH
(SEQ ID NO: 8)
TC14012 H-Arg-Arg-Nal-Cys-Tyr-Cit-Lys-_{D}-Cit-Pro-Tyr-Arg-Cit-Cys-Arg-NH₂
(SEQ ID NO: 9)
TE14005 H-Arg-Arg-Nal-Cys-Tyr-Arg-Lys-_{D}-Glu-Pro-Tyr-Arg-Cit-Cys-Arg-OH
(SEQ ID NO: 10)
4F-benzoyl-TN14003 4-fluorobenzoyl-Arg-Arg-Nal-Cys-Tyr-Cit-Lys-_{D}-Lys-Pro-Tyr-Arg-Cit-
Cys-Arg-NH₂

### Diagnosis of Musculoskeletal Disorders

Examples of musculoskeletal disorders include: sprains, strains, tendonitis, tenosynovitis, fibromyalgia, osteoarthritis, rheumatoid arthritis, polymyalgia rheumatica, bursitis, acute and chronic back pain and osteoporosis, specifically rotator cuff tendonitis, epicondylitis olecranon bursitis, trochanteric bursitis, or patellar tendonitis. Pain is the most common symptom and is frequently caused by injury or inflammation. Besides pain, other symptoms such as stiffness, tenderness, weakness and swelling or deformity of affected parts are manifestations of musculoskeletal disorders. In some embodiments of the present invention, a musculoskeletal disorder is selected from the group consisting of Bursitis (subacromial, olecranon, trochanteric, ischial, pre-patellar), Tendonitis (rotator cuff, biceps, patellar, achilles, pes anserine, flexor carpi radialis, Tenosynovitis (dequervains, flexor tendon, trigger finger), Epicondylitis (tennis elbow, golfers elbow), Impingement Syndrome, Rotator cuff disease (tendonitis, rotator cuff tear) Fasciitis (plantar fasciitis, iliotibial band syndrome), Enthesopathies, and Tendon injury, rupture, or tear (rotator cuff, biceps, triceps, pectoralis, achilles, patellar, elbow and finger flexor and extensor tendons, hamstring, quadriceps, peroneal, toe digital flexor and extensor tendons). (Blaine et al. J. Shoulder and Elbow Surgery, 14: 84S-89S (2005)).

### Methods of Treatment

Bursitis and tendonitis are some of the most common musculoskeletal complaints. The etiology is likely multifactorial and includes both mechanical and biologic factors. Bursitis is characterized by chronic inflammation. In addition to the up-regulation of several cytokines (TNF α, IL-1β, IL-6) and cyclooxygenases (COX-1, COX-2), the data indicates that SDF-1α, a potent pro-inflammatory chemokine that mediates the bursa and tendon cell's response to mechanical or biochemical signals.

SDF-1alpha and its receptor CXCR4 play an important role in cell migration, embryonic development, cancer, and human immunodeficiency virus infection. CXCR4 is the major HIV type 1 (HIV-1) co-receptor required for viral entry into the host cells. Inhibitors to the SDF-1α receptor CXCR4 have been designed and proposed for clinical use in the prevention and treatment of HIV infection. Thus, these inhibitors are useful in the treatment of musculoskeletal inflammation including tendonitis and bursitis.

While injection of corticosteroid medication is a common outpatient procedure, both corticosteroids and non-steroidal anti-inflammatory (NSAIDs) have systemic risks, and may inhibit or impair tendon healing. Therefore, the methods and inhibitors described herein have significant clinical value. By targeting inhibition of the SDF-1α, these pharmacologic agents are used to treat many common and painful conditions with fewer side effects than NSAIDs and steroids.

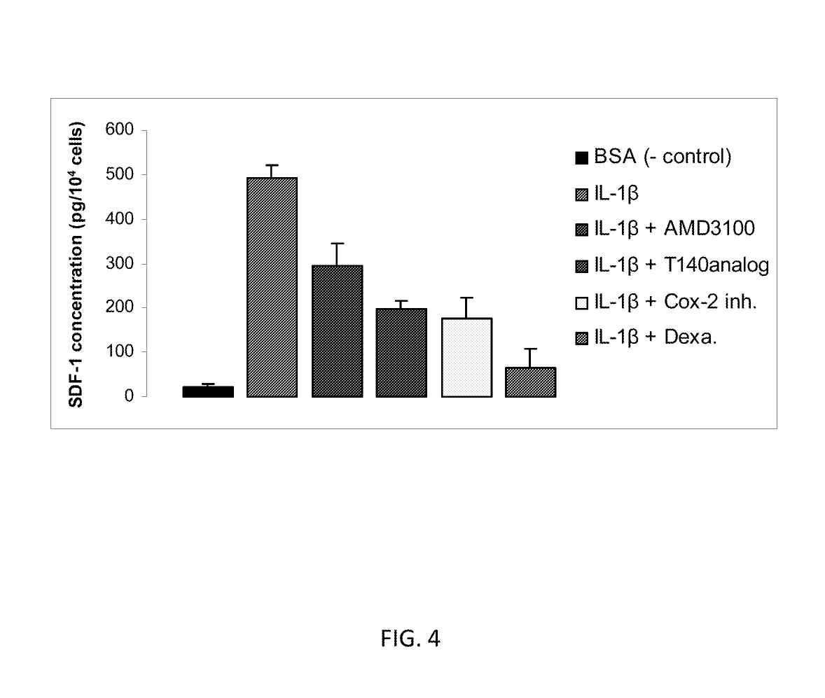

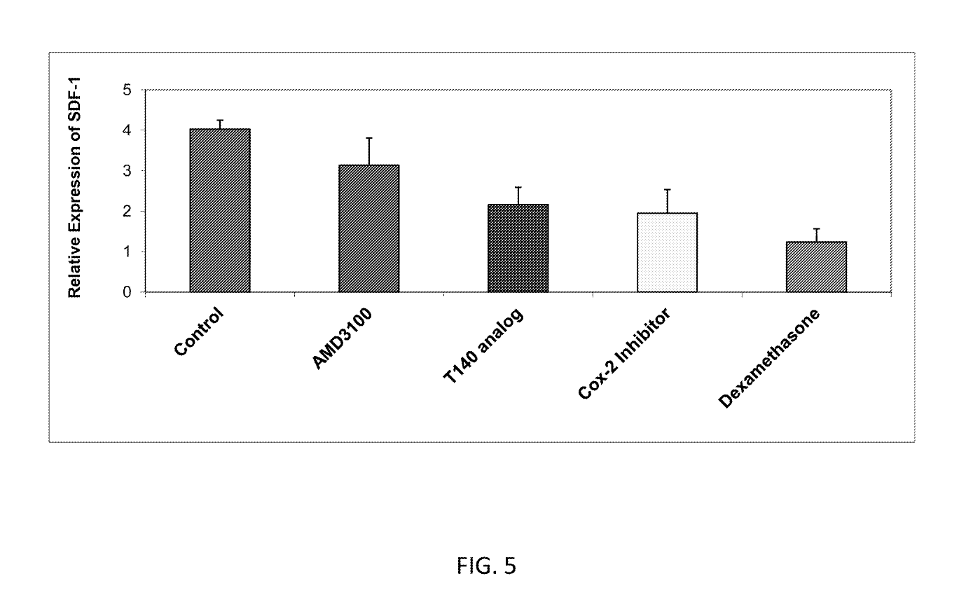

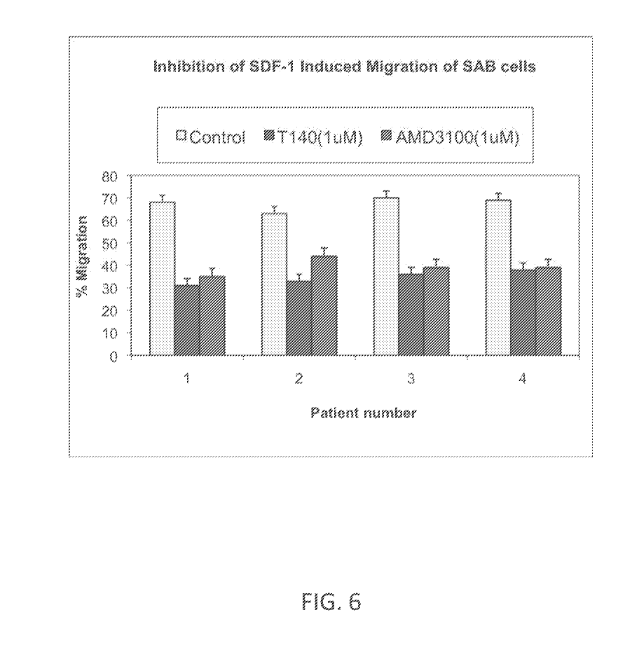

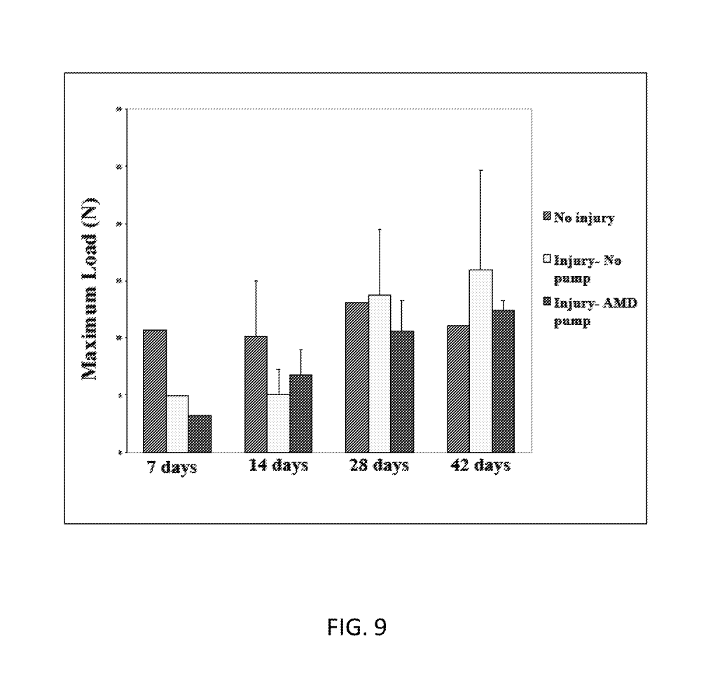

SDF-1α protein and mRNA expression were decreased in subacromial bursa cells treated with CXCR4 inhibitors (FIGS. 4-5). Although maximal inhibition (87%) occurred in bursa cells treated with dexamethasone, both CXCR4 inhibitors (T140 analog & AMD3100) significantly decreased both SDF-1α protein and mRNA expression in these cells. T140 analog was the more potent inhibitor, decreasing SDF-1α protein production by 60 percent. These results were comparable to those seen with COX-2 inhibition (64%). While T140 analog significantly decreased SDF-1α mRNA expression, AMD3100 decreased SDF-1α protein production by 40 percent but caused only a slight decrease in SDF-1α mRNA expression. Bursal cell migration in response to SDF-1 stimulation was decreased in the presence of both CXCR4 inhibitors (T140 analog and AMD3100). These results were comparable to those seen with COX-2 inhibitors and dexamethasone and were seen consistently in bursa cells cultured from multiple patients. Previously known non-selective agents (NSAIDs, corticosteroids or cortisone injections) inhibit the healing of tendons and lead to significant local (impaired rotator cuff tendon healing) and systemic (cardiovascular and gastrointestinal) adverse events. However, CXCR4 inhibitors of the present invention promote the healing of tendons (FIG. 9).

Accordingly, provided herein are methods for treating or ameliorating a musculoskeletal disorder in a subject in need of by administering to the subject a compound that inhibits or reduces binding of SDF-1α and/or CXCR4 or reduces expression of SDF-1α and/or CXCR4.

The term “treating” as used herein refers to alleviate of at least one symptom of the disease, disorder or condition. The term encompasses the administration and/or application of one or more compounds described herein, to a subject, for the purpose of providing management of, or remedy for a condition. “Treatment” for the purposes of this disclosure, may, but does not have to, provide a cure; rather, “treatment” may be in the form of management of the condition. When the compounds described herein are used to treat a musculoskeletal disorder, “treatment” includes partial or total reduction of inflammation associated with the disorder.

For example, the compound inhibits or binds to SDF-1α, therefore preventing or reducing the binding of SDF-1α and CXCR4. Alternatively, the compound inhibits or binds to CXCR4, which leads to prevention or reduction in the binding of SDF-1α and CXCR4. An exemplary CXCR4 blocker is a small molecule inhibitor, such as AMD 3100. Another example of a CXCR4 blocking agent is a peptide inhibitor, such as a T140 analog.

Musculoskeletal disorders to be treated include:
Bursitis (subacromial, olecranon, trochanteric, ischial, pre-patellar)
Tendonitis (rotator cuff, biceps, patellar, achilles, pes anserine, flexor carpi radialis
Tenosynovitis (dequervains, flexor tendon, trigger finger)
Epicondylitis (tennis elbow, golfers elbow)
Impingement Syndrome
Rotator cuff disease (tendonitis, rotator cuff tear)
Fasciitis (plantar fasciitis, iliotibial band syndrome)
Enthesopathies
Tendon injury, rupture, or tear (rotator cuff, biceps, triceps, pectoralis, achilles, patellar, elbow and finger flexor and extensor tendons, hamstring, quadriceps, peroneal, toe digital flexor and extensor tendons)
Inflammatory condition that affects the musculoskeletal system

Preferably, the musculoskeletal disorders to be treated include bursitis and tendonitis, as well as rotator cuff tendonitis, achilles tendonitis ankle, patellar tendonitis, tennis elbow, trochanteric bursitis, epicondylitis olecranon bursitis, subacromial impingement syndrome, and subacromial inflammation.

### Therapeutic Administration

A pharmaceutical composition of the invention is formulated to be compatible with its intended route of administration. Methods of administering inhibitors for treatment of musculoskeletal disorders are known in the art. Generally, procedures are performed in the doctor's office. Local administration is often carried out by injection, e.g., similar to a procedure used for cortisone injections.

The procedure for injection therapy is uncomplicated and well established. The technique is similar for muscle, periarticular, or articular injections. In general, three techniques are utilized: anatomic landmarks (e.g., by palpation), fluoroscopic guidance, and ultrasound guidance. Optionally, a local anesthetic (e.g., lidocaine) is injected prior to the injection of an inhibitor agent. In some cases, the anesthetic is mixed with the active agent (inhibitor). Generally, a small, 25-gauge needle or a larger-bore needle (21-22 gauge) is used for the injection depending on the viscosity of the agent to be administered.

For periarticular injections, the injection is generally accomplished in small droplets around the area of inflammation. Multiple injections may be required to infiltrate several centimeters of the tendon and muscle. Joint injections are accomplished by inserting the needle directly into the joint using standard methods (e.g., Simons et al., 1998, Myofascial Pain and Dysfunction: The Trigger Point Manual. 2nd ed. Baltimore, Md.: Lippincott Williams & Wilkins; 1998). Multiple injections over time (e.g., 3 injections per joint per calendar year) may be required for comprehensive treatment of the patient. A description of exemplary joint injection techniques follows.

For the shoulder, injection of the subacromial space for the treatment of rotator cuff tendinitis and shoulder impingement syndrome is a common procedure. For example, a posterolateral approach is used (Bell et al., 2005, Int J Clin Pract. 59:1178-86). Briefly, the steps include: palpate the posterior tip of the acromion, and insert the needle into the space between the acromion and the head of the humerus; angle the needle anteriorly toward the coracoid process; once in the space, draw back on the syringe to ensure that the needle is not in a vascular structure. Resistance during delivery of the medication should be minimal.

Knee injection procedures are also well known, e.g., (Courtney et al., 2005, Best Pract Res Clin Rheumatol. 19:345-69). Briefly, the steps include: palpate the inferior medial aspect of the patella, and insert the needle into the space between the patella and femur, parallel to the inferior border of the patella; angle the needle to the center of the patella; aspirate any fluid before performing the injection; deliver the inhibitory agents and withdraw the needle.

For the hand and wrist (e.g., for the treatment of carpal tunnel syndrome), steps include: with the palmar surface of the hand facing upward, inject just proximal to the flexor crease and between the palmaris longus tendon and the flexor carpi radialis tendon, the needle enters the skin at a 45° angle and is aimed toward the tip of the middle finger; advance the needle 1 to 2 cm until resistance is felt; withdraw the needle slightly, and inject the medication.

For the elbow (e.g., for treatment of lateral epicondylitis), an exemplary technique is described in Torp-Pedersen et al., 2008, Br J Sports Med. Mar. 4 2008. Steps include: palpate the lateral epicondyle, with the arm faced palm down and elbow flexed to about 45°, identify a point about 1 cm superior and 1 cm distal to the lateral epicondyle; inject the medication into the point of maximum tenderness; repeatedly withdraw and redirect the needle to infiltrate the area.

For the hip (e.g., for treatment of bursitis of the greater trochanter), an exemplary method includes the following steps: identify the point of maximal tenderness, which typically is over the posteroinferior edge of the greater trochanter; advance the needle until it gently contacts bone; withdraw the needle about 0.25-0.5 cm, and administer a partial injection; additional medication is infiltrated into the surrounding area in a fan-shaped pattern.

Other examples of routes of administration include parenteral, e.g., intravenous, intradermal, subcutaneous, oral (e.g., inhalation), or transdermal (i.e., topical). Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. For example an inhibitor of the present invention is administered intraoperatively, arthroscopically, or by local direct injection into the affected tissue.

For reduction of inflammation associated with musculoskeletal disorders such as bursitis or tendonitis, the inhibitor compound is administered as an injection of a single 1.2 ml dose of a 20 mg/ml solution (24 mg). Alternatively, the inhibitor compound is administered using an average dose of 0.1 mg/kg/day, 0.2 mg/kg/day, 0.34 mg/kg/day, 0.5 mg/kg/day, 1 mg/kg/day, 1.5 mg/kg/day, about 2 mg/kg/day, about 5 mg/kg/day, or more if necessary. In another example, the inhibitor compound is administered 1-100 mg per injection. In a further example, the inhibitor compound is administered 0.24 mg/kg per dose per day. In an alternative example, the inhibitor compound is administered as a single intravenous infusion at the highest dose of 80 μg/kg.

Examples of dosing regimens that can be used in the methods of the invention include, but are not limited to, daily, three times weekly (intermittent), weekly, or every 14 days. In certain embodiments, dosing regimens include, but are not limited to, monthly dosing or dosing every 6-8 weeks.

The formulations of the present invention contain an amount of CXCR4 inhibitors that is effective for the intended use. The formulations of CXCR4 inhibitors may be prepared as a liquid or in a solid form such as a powder, tablet, pill or capsule. Liquid formulations may take such forms as suspensions, solutions or emulsions in oily or aqueous vehicles, and may contain formulatory agents such as suspending, stabilizing and/or dispersing agents. In one embodiment, the formulation is an aqueous solution. In another embodiment, the final formulation is lyophilized. In other embodiments, the formulation comprises a colloidal drug delivery system. Such drug delivery systems include, for example, liposomes, albumin microspheres, microemulsions, nano-particles and nanocapsules.

### Kits

A CXCR4 inhibitor may, if desired, be presented in a kit (e.g., a pack or dispenser device) which may contain one or more unit dosage forms containing the CXCR4 inhibitor. The pack may for example comprise metal or plastic foil, such as a blister pack. The pack or dispenser device may be accompanied by instructions for administration. Compositions comprising a CXCR4 inhibitor of the invention formulated in a compatible pharmaceutical carrier may also be prepared, placed in an appropriate container, and labelled for treatment of an indicated condition. Instructions for use may also be provided.

Also provided herein are kits comprising a plurality of detection reagents that detect the expression of SDF-1α and/or CXCR4. For example, the detection reagent is primers, antibodies or fragments thereof, polypeptide or aptamers. The kit may contain in separate containers an aptamer or an antibody, control formulations (positive and/or negative), and/or a detectable label such as fluorescein, green fluorescent protein, rhodamine, cyanine dyes, Alexa dyes, luciferase, radiolabels, among others. Instructions (e.g., written, tape, VCR, CD-ROM, etc.) for carrying out the assay may be included in the kit. The assay may for example be in the form of a PCR, Western Blot analysis, Immunohistochemistry (IHC), immunofluorescence (IF) and Mass spectrometry (MS) as known in the art.

### EXAMPLES

The invention now being generally described, it will be more readily understood by reference to the following examples, which are included merely for purposes of illustration of certain aspects and embodiments of the present invention, and are not intended to limit the invention.

### Example 1

### Methods and Materials

### Immunohistochemistry (IHC) of Bursal Specimens

Human subacromial bursal biopsy specimens were obtained under written informed consent. Fresh subacromial bursal tissues were obtained from patients undergoing shoulder surgery. Tissue sections were then fixed in 10% formalin, decalcified, and embedded in paraffin. Sections were stained for Hematoxylin/Eosin (HE). The remainder were labeled with primary monoclonal antibodies against IL-1β, IL-6, SDF-1α and CXCR4. Several control slides from each set were made using only phosphate buffered solution (PBS).

### Cell Cultures and Characterization

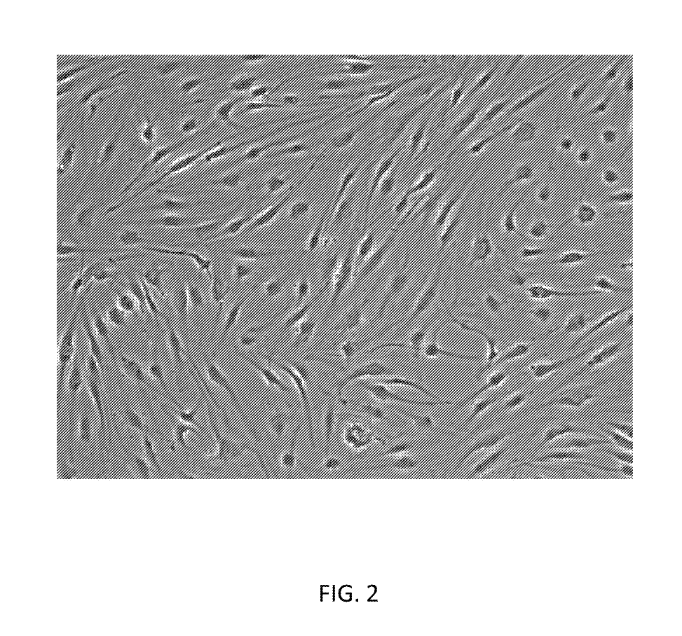

Human subacromial bursal biopsy specimens were obtained as above. Tissues were washed with Hank's balanced salt solution (Gibco), minced with scissors, and resuspended in Dulbecco's minimum essential medium (DMEM, Gibco) containing 1 mg/ml collagenase, 0.15 mg/ml DNase, and 0.15 mg/ml hyaluronidase. The tissue was enzymatically digested for 2 hours at 37° C. and then passed through sterile gauze to remove any undigested fragments. Cells were seeded in 75 cm2 flasks with DMEM supplemented with 10% fetal bovine serum (FBS), 100 U/ml penicillin, 100 μg/ml streptomycin, and 0.1% fungizone (amphotericin B) and grown at 37° C. in a humidified atmosphere of 5% CO2 and 95% air. Subcultures were performed with Trypsin-EDTA treatment (Gibco). In these conditions, 100% of the adherent cells had a synovial fibroblast-like phenotype (FIG. 2).

To detect surface antigens, cells isolated from human shoulder tissue were first harvested using Trypsin-EDTA and washed three times in ice cold FACS buffer (1×PBS, 5% FBS and 0.1% sodium azide.) Antibodies against the human antigens CD44 and CD 106 were obtained from BD Biosciences (Carlsbad, Calif.). Cells were stained with 10 μg of either fluorescein isothiocyanate-conjugated CD106 or phycoerythrin-conjugated CD44. The suspensions were incubated for 30 min. at 4° C. Following incubation, cells were washed 3 times with ice cold FACS buffer. Suspensions were fixed with 10% formalin (1:1) and were stored in the dark overnight at 4° C. Analysis was performed by flow cytometry using a FACSVantage (BD Biosciences).

### Protein Expression

Subacromial bursal cells were cultured as above. After the first passage, cells were plated into 6-well plates and grown to 85% confluence. Wells were then treated (in triplicate) with IL-1β 20 ng/mL and respective inhibitors (AMD3100, T140 analog, dexamethasone or NSAID). After 24 hours, supernatants were collected and assayed for SDF-1α expression using Quantikineâ Human SDF-1α enzyme immunoassay kit (R & D System) according to the manufacturer's instructions. Standard curves were reconstituted using SDF-1α protein. The optical density of each well was determined using a microplate reader set to 450 nm wavelength. Statistical analysis was performed using the Student's t test. Statistical significance was present when p<0.05.

### Gene Expression

Subacromial bursal cells were cultured as above. First-passaged cells were treated with IL-1β and respective inhibitors as above. Total RNA was isolated using Qiagen RNeasy Mini Kit (Qiagen, Valencia, Calif.). First-strand cDNA was synthesized using 1 μg of total RNA and Superscript III™ First-Strand Synthesis System (Invitrogen). The reaction mixture was incubated at 50° C. for 50 min followed by a denaturation step of 5 min at 85° C. SDF-1 mRNA was quantified with SDF-1 specific primers SDF-1FOR (5′-GATTGTAGCCCGGCT GAAGAA-3′) (SEQ ID NO: 11) and SDF-1BACK (5′-ACCTGGTCCTCATGGTTAAGGC-3′) (SEQ ID NO: 12). All PCR were performed in 25-μl reactions on a DNA Engine Opticon™ 2 Detection System (MJ Research, Hercules, Calif.) for 40 cycles in which each cycles consisted of 15 sec at 95° C., 15 sec at 60° C., and 20 sec at 72° C. Results were standardized to GAPDH housekeeping gene. Statistical analysis was performed using the student's t-test.

### Example 2

### CXCR4 Blockade Inhibits SDF-1 Expression and Cell Migration in Human Subacromial Bursa Cells

Described herein are cellular and biochemical events that occur in the process of subacromial inflammation and rotator cuff syndrome. Inflammatory mediators have been implicated in causing cytokine-induced tendonitis, and subacromial bursitis is characterized by chronic inflammation in the subacromial bursa. In addition to the up-regulation of several cytokines (TNFa, IL-1β, IL-6) and cyclooxygenases (COX-1, COX-2), SDF-1α, a potent pro-inflammatory chemokine, was found to mediate the bursal cell's response to mechanical or biochemical signals.

Chemokines are a soluble peptide family that regulate cell movement, proliferation, and differentiation, exerting their effects through a family of transmembrane G-protein coupled receptors. SDF-1α is an 8 KDa peptide originally isolated from a bone marrow stromal cell line. It activates a variety of primary cells through binding to its receptor CXCR4. SDF-1α and its receptor CXCR4 in the subacromial bursa were found to play a role in mediating chemokine-induced subacromial inflammation that is seen in human rotator cuff disease.

IL-1β is present in the human subacromial bursa and may also play a significant role in the inflammation seen in rotator cuff disease. [18,19] IL-1β directly stimulates SDF production in several cell types, however it has been shown that the signaling mechanisms between IL-b and SDF may vary among different cells. [29,36] IL-1β stimulates the expression of SDF-1α in bursal cells, however the mechanisms of this signaling and the relative importance of each to subacromial inflammation have not been characterized. [23]

SDF-1α and its receptor CXCR4 have been shown to be involved in human immunodeficiency virus infection. [36-38,43,44,48,49] CXCR4 is the major HIV type 1 (HIV-1) co-receptor required for viral entry into the host cells. Inhibitors to the SDF-1α receptor CXCR4 have been designed and proposed for clinical use in the prevention and treatment of HIV infection. [23, 42] Similar to SDF-1α receptor inhibitors, IL-1β receptor inhibitors have been designed and are in clinical use in the treatment of rheumatoid arthritis and autoimmune disease. [39-41,45]

Local cytokines in the subacromial bursa, specifically IL-1β and IL-6, may directly stimulate the bursal synoviocyte to express SDF-1α. The data described herein has demonstrated that IL-1β induced SDF-1 production by the subacromial bursal cell was inhibited by SDF-1 receptor (CXCR4) inhibitors AMD3100 and T140 analog; and that bursal cell activity, as measured by bursal cell migration, was modulated by these inhibitors.

### Characterization of Bursal Cells

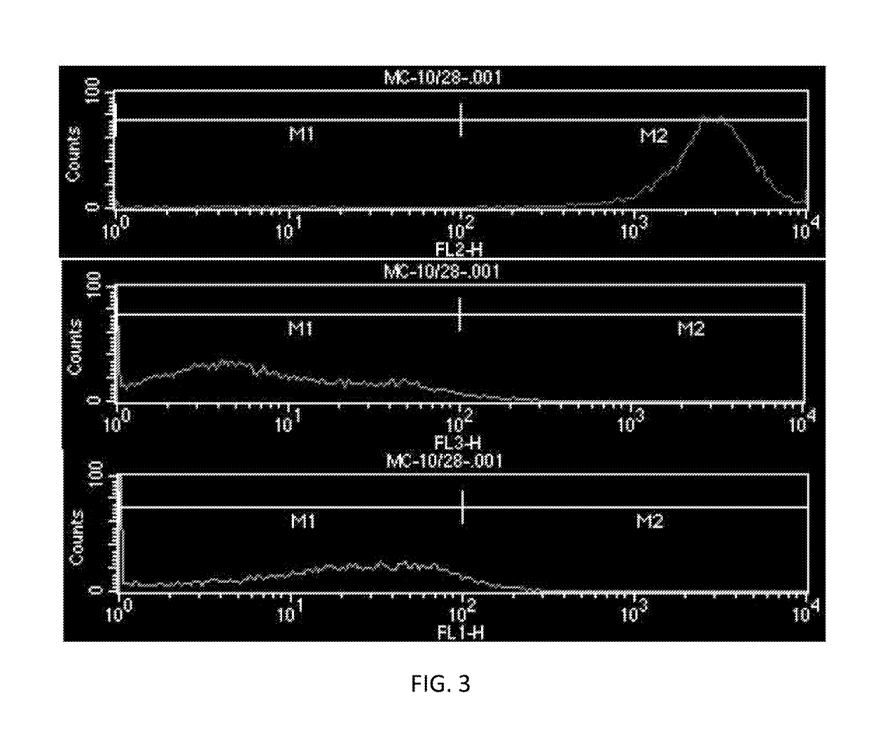

Flow cytometric analysis showed that human subacromial bursal cells in culture are of the fibroblastic synoviocyte lineage. More than 95% of harvested subacromial bursal cells expressed CD44 antigens, whereas less than 2% expressed CD106 antigens (FIG. 3). These results coincide with cell surface markers used to detect human bursal cells.

### Chemokines are Expressed in Human Subacromial Bursa.

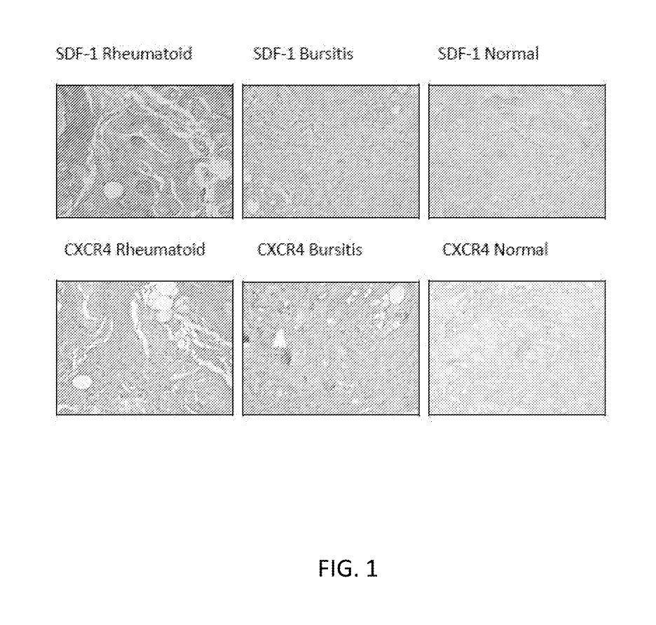

It has been demonstrated that chemokines (SDF-1), cytokines (IL-1β and IL-6) and their receptors (CXCR4) are expressed in the human subacromial bursa both in tissue sections obtained from surgery and in cell cultures of bursal synoviocytes. SDF-1mRNA and protein are increased in patients with bursitis as compared to control specimens. Furthermore, immunohistochemical analysis of these specimens indicates high levels of expression of these mediators (FIG. 1) which are increased above normal specimens but reduced as compared to rheumatoid synovium.

IL-1β Induces Expression of SDF-1 mRNA and Protein in Cell Cultures.

SDF-1 expression was induced by stimulating bursal cells with IL-1β. SDF-1 levels were measured in cell supernatants and mRNA levels were tested using real-time RT-PCR at various time points. SDF-1 expression increased with time and occurred in a dose-dependent fashion. (FIG. 2) IL-6 did not stimulate the cells to produce SDF-1. (FIG. 3) These experiments were reproducible with cells grown from several different tissue samples.

Gene Expression

Quantitative reverse-transcriptase (RT)-PCR demonstrated that SDF-1α mRNA expression was increased in bursal cells treated with IL-1β compared to controls (p<0.05). An increase in expression was first observed after 6 hours and continued to increase thereafter. Maximal stimulation was seen at 24 hours (p<0.05) of induction with IL-1β, exhibiting a nearly five-fold increase in SDF-1α expression (p<0.05).

Protein Expression

SDF-1α production was increased in subacromial bursal cells stimulated with IL-1β. Protein production began just 6 hours after induction and increased thereafter. Maximal stimulation occurred after 48 hours of incubation with IL-1β. IL-1β stimulated the production of SDF-1α in a dose-dependent fashion. Maximal stimulation (630 pg/10⁴ cells) occurred with exposure to 20 ng/ml IL-1β. (p<0.05)

### IL-1β Induced SDF-1 Expression in Human Subacromial Bursal Cells is Inhibited by CXCR4 Inhibitors, NSAIDs, and Dexamethasone.

Cultured human subacromial bursal cells were treated with 20 ng/mL of IL-1β. After 24 hrs, media was changed and inhibitors were added. Supernatants were collected after 48 hrs and assayed for SDF-1α expression using ELISA. Total RNA was isolated using Qiagen RNeasy Mini Kit (Qiagen, Valencia, Calif.) and RT-PCR performed to assess SDF-1 mRNA expression. SDF-1α expression was decreased in the supernatants of bursal cells treated with each inhibitor. Maximal inhibition (87%) occurred with dexamethasone. The cox-2 inhibitor and T140 analog showed similar patterns of inhibition, decreasing expression by 64% and 60%, respectively. AMD3100, a CXCR4 receptor antagonist, decreased SDF-1α expression by 40%. (FIG. 4) While T140 analog significantly decreased SDF-1α mRNA expression, AMD3100 decreased SDF-1α protein production by 40 percent but caused only a slight decrease in SDF-1α mRNA expression. (FIG. 5)

The induction and regulation of SDF-1α plays a central role in the pathophysiology of subacromial impingement. However, prior to the invention, the mechanisms by which SDF-1α is induced and regulated were not understood. SDF-1α is elevated in the subacromial bursa of patients with rotator cuff disease as compared to controls, and can be induced by mechanical stimuli. IL-1β is present in the human subacromial bursa and may also play a significant role in the inflammation seen in rotator cuff disease. No prior studies have demonstrated the ability of IL-1β to induce expression of SDF-1 in the subacromial bursa.

Using an in vitro model of subacromial impingement, the present study demonstrates that cytokines may induce expression of SDF-1α in cultured subacromial bursal cells. IL-1β was a more potent inducer of SDF-1α than IL-6, causing a more than five-fold increase in SDF-1α gene and protein expression in cultured bursal cells. Induction occurred early, with gene expression increased at six hours and reaching maximum expression levels at 24 hours post stimulation. The response was dose-dependent, and the resultant levels of SDF-1 levels were at or above physiologic levels (25-50 ng/ml). Immunohistochemistry studies further demonstrated that these cytokines, chemokines and their receptors are expressed in human subacromial bursal specimens. IL-1β, IL-6 and SDF-1α were all present in bursal specimens, with expression concentrated in the bursal synovial cells. The SDF-1α receptor, CXCR4, was also present in these specimens.

The data described herein demonstrated inflammatory mediator expression and chronic inflammation in the subacromial bursa. Up-regulation of several cytokines (TNFα, IL-1β, IL-6) and cyclooxygenases (COX-1, COX-2) were shown in IHC analysis of bursal specimens, and this expression was increased in patients with subacromial bursitis and compared to controls. A novel role SDF-1α, a potent pro-inflammatory chemokine, was identified. It mediates the bursal cell's response to mechanical or biochemical signals.

Prior to the invention, there was little or no information on IL-1β/SDF-1α signaling in subacromial bursa synoviocytes. The data described herein provide initial information on this important pathway in these cells.

Based on these findings, one pathway of inflammation in the human subacromial bursa involves the interaction between IL-1β and SDF-1. IL-1β is produced by rotator cuff tenocytyes, subacromial bursa cells, or inflammatory cells in response to rotator cuff injury or other mechanical stimulation. Secreted IL-1β may then serve in an autocrine or paracrine fashion to stimulate the production of SDF-1α by bursal synoviocytes, leading to chronic and persistent inflammation in the subacromial bursa. This in vitro study demonstrates significant clinical relevance in these findings. Subacromial impingement syndrome and rotator cuff disease is the most common presenting diagnosis in shoulder and elbow surgery. The non-selective agents (NSAIDs and corticosteroids) currently used in its treatment may lead to significant local (impaired rotator cuff tendon healing) and systemic (cardiovascular and gastrointestinal) adverse events.

These data provide further information on the biochemical signals which mediates the molecular pathophysiology of rotator cuff disease. With the current availability of inhibitors to both IL-1β and SDF-1α which are in clinical use for other indications, these findings have significant clinical relevance for the treatment of subacromial inflammation and rotator cuff disease and other musculoskeletal disorders. Further studies are needed to investigate the effects of these various inhibitors on subacromial inflammation and rotator cuff healing.

### Example 3

### SDF-1α Plays a Central Role in the Pathophysiology of Subacromial Impingement

### Interleukin-1β Stimulates Stromal Derived Factor-1α Expression In Human Subacromial Bursa

Chemokines produced by synoviocytes of the subacromial bursa are up-regulated in subacromial bursitis and rotator cuff disease. Studies were carried out to determine whether SDF-1α production in bursal synoviocytes is induced by local cytokines such as interleukin IL-1β and IL-6.

Subacromial bursa specimens were obtained from patients undergoing shoulder surgery. Bursal specimens were stained with anti-human antibodies to IL-1, IL-6 and SDF-1α by immunohistochemistry and compared to normal and rheumatoid controls. Bursal cells were also isolated from specimens and cultured. Early-passaged cells were then treated with cytokines (IL-β and IL-6) and SDF-1α expression was measured by ELISA and RT-PCR.

SDF-1α, IL-1β and IL-6 were found to be expressed at high levels in bursitis specimens from human subacromial bursa compared to normal controls. In cultured bursal synoviocytes, there was a dose-dependent increase in SDF-1α production in the supernatants of cells treated with IL-1β. SDF-1α mRNA expression was also increased in bursal cells treated with IL-1β. IL-6 caused a minimal but not statistically significant increase in SDF-1α expression.

SDF-1, IL-1β, and IL-6 are expressed in the inflamed human subacromial bursal tissues in patients with subacromial bursitis. In cultured bursal synoviocytes, SDF-1α gene expression and protein production is stimulated by IL-1β. IL-1β produced by bursal synoviocytes and inflammatory cells in the human subacromial bursa is an important signal in the inflammatory response that occurs in subacromial bursitis and rotator cuff disease.

### Subacromial Impingement Syndrome

Subacromial impingement syndrome is a common cause of shoulder pain. While mechanical impingement is considered the major cause of inflammation in the subacromial bursa, there is little information on the cellular and biochemical events that define this process. Increases in afferent nerve endings and their products have been identified in inflamed subacromial bursa, and several studies have identified inflammatory mediators in both the glenohumeral joint and the subacromial space, implicating their role in cytokine-induced tendonitis.

Subacromial bursitis is characterized by chronic inflammation in the subacromial bursa and rotator cuff disease is associated with the increased expression of inflammatory cytokines including stromal cell-derived factor 1 (SDF-1α; CXCL12) and human interleukins IL-1β and IL-6 in the subacromial bursa. SDF-1 is an important mediator in the bursal cell's response to biochemical signals. However, prior to the invention, little was known about the mechanisms of induction of SDF-1 expression in the subacromial bursal cell.

The data described herein indicates that local cytokines in the subacromial bursa, specifically IL-1β and IL-6, directly stimulate the bursal synoviocyte to express SDF-1α.

### SDF-1α Plays a Central Role in the Pathophysiology of Subacromial Impingement

Prior to the invention, the mechanisms by which SDF-1α is induced and regulated were not completely understood. A number of inflammatory mediators are present and may play a role in inflammation in the subacromial bursa, e.g., IL-1, IL-6, TNF, MMPS, VEGF, and SDF-1. SDF-1α is elevated in the subacromial bursa of patients with rotator cuff disease as compared to controls, and can be induced by mechanical stimuli. IL-1β is present in the human subacromial bursa and may also play a significant role in the inflammation seen in rotator cuff disease. No prior studies have demonstrated the ability of IL-1β to induce expression of SDF-1α in the subacromial bursa.

Using an in-vitro model of subacromial impingement, the present study demonstrates that cytokines induce expression of SDF-1α in cultured subacromial bursal cells. IL-1β was a more potent inducer of SDF-1α than IL-6, causing a more than five-fold increase in SDF-1α gene and protein expression in cultured bursal cells. Induction occurred early, with gene expression increased at six hours and reaching maximum expression levels at 24 hours post stimulation. The response was dose-dependent, and the resultant levels of SDF-1α levels were at or above physiologic levels (25-50 ng/ml). Immunohistochemistry studies further demonstrated that these cytokines, chemokines and their receptors are expressed in human subacromial bursal specimens. IL-1β, IL-6 and SDF-1α were all present in bursal specimens, with expression concentrated in the bursal synovial cells. The SDF-1α receptor, CXCR4, was also present in these specimens.

The data described herein indicate that one pathway of inflammation in the human subacromial bursa involves the interaction between IL-1β and SDF-1α. IL-1β is produced by rotator cuff tenocytyes, subacromial bursa cells, or inflammatory cells in response to rotator cuff injury or other mechanical stimulation. Secreted IL-1β then serves in an autocrine or paracrine fashion to stimulate the production of SDF-1α by bursal synoviocytes, leading to chronic and persistent inflammation in the subacromial bursa. Inhibitors of secreted IL-1β and/or SDF-1α are useful to reduce or inhibit inflammation associated with these conditions.

### Clinical Relevance

Subacromial impingement syndrome and rotator cuff disease is the most common presenting diagnosis in shoulder and elbow surgery. However, the non-selective agents (NSAIDs and corticosteroids) currently used in its treatment lead to significant local (impaired rotator cuff tendon healing) and systemic (cardiovascular and gastrointestinal) adverse events. The invention overcomes the drawbacks of the earlier methods. Administration of compounds that block or inhibit SDF-1α/CXCR4 signaling or that inhibit IL-1β/SDF-1α signaling reduce inflammation associated with bursitis and tendonitis, as well as other musculoskeletal disorders described herein without the adverse side effects associated with NSAIDs and corticosteroids.

### Example 4

### Novel Anti-Inflammatory Agent and CXCR$ Inhibitor does not Impede Rotator Cuff Tendon Healing

Rotator cuff tendonitis and subacromial inflammation are mediated in part by SDF-1a expression in the subacromial bursa. A potential target for inhibition of SDF-1a mediated inflammation includes the SDF-1 receptor, CXCR4. In the instant invention, we have demonstrated that SDF-1a expression and subacromial bursa cell activity could be inhibited with a clinically available CXCR4 inhibitor, AMD 3100. Further, we designed the following study to determine the effects of this agent on rotator cuff healing in an animal model. The study demonstrates that the CXCR4 inhibitor, AMD 3100, reduced inflammation in the subacromial bursa in rat shoulder with experimentally induced rotator cuff injury, but did not show any adverse effect on tendon healing.

### Methods

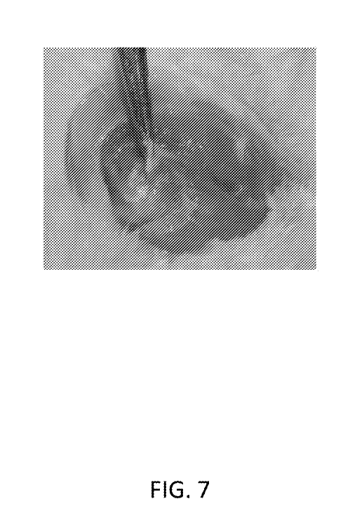

58 male Lewis rat shoulders underwent surgery using an IACUC approved protocol. The infraspinatus tendon was transected at its insertion to create a tendon defect (FIG. 7). Osmotic minipumps (Alzet) containing either AMD 3100 or Phosphate Buffered Saline (PBS) were inserted into a subcutaneous pocket developed via blunt dissection. Animals were sacrificed at 3, 7, 14, 28 or 42 days. Eight uninjured rat shoulders were used as controls.

### Mechanical Testing Protocol

Preconditioned for 5 cycles to 0.38 mm displacement in tension at a rate of 0.1 mm/s

Stress relaxation test for 300 s duration at 0.38 displacement in tension

Return to 0.2 N tension followed by a 300 s latency period

Tension to failure at a displacement rate of 0.1 mm/s

### Results

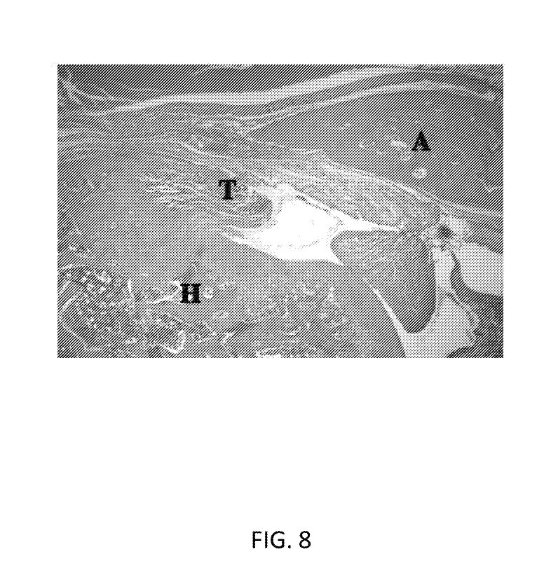

Maximum load in the infraspinatus bone-tendon complex was significantly reduced at 3 days and persisted until 28 days post injury. By 28 days, maximum load approached that of uninjured tendons in all groups. No statistically significant differences in maximum load were observed in the AMD 3100 group as compared to controls at any time-point (FIGS. 8 and 9).

CXCR4 inhibitors (such as AMD 3100) significantly decrease inflammation in human subacromial bursa cells, providing a novel anti-inflammatory pathway for the treatment of subacromial bursitis and rotator cuff disease. In this example, we have further shown in a rat model of rotator cuff disease that AMD 3100 does not inhibit rotator cuff healing based on histological and biomechanical testing. CXCR4 inhibitors such as AMD 3100 therefore present a new means to reduce inflammation, with fewer side effects, in the treatment of rotator cuff disease and other musculoskeletal disorders.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### REFERENCES

1. Bigliani L U, Levine W N. Subacromial impingement syndrome. J Bone Joint Surg Am 1997; 79:1854-68.
2. Blades M C, Ingegnoli F, Wheller S K, et al. Stromal cell-derived factor 1 (CXCL12) induces monocyte migration into human synovium transplanted onto SCID mice. Arthritis Rheum 2002; 46:824-36.
3. Blaine T A, Kim Y S, Voloshin I, et al. The molecular pathophysiology of subacromial bursitis in rotator cuff disease. J Shoulder Elbow Surg 2005; 14:84 S-89S.
4. Bradfield P F, Amft N, Vernon-Wilson E, et al. Rheumatoid fibroblast-like synoviocytes overexpress the chemokine stromal cell-derived factor 1 (CXCL12), which supports distinct patterns and rates of CD4+ and CD8+ T cell migration within synovial tissue. Arthritis Rheum 2003; 48:2472-82.
5. García-Moruja C, Alonso-Lobo J M, Rueda P, et al. Functional characterization of SDF-1 proximal promoter. J Mol Biol 2005; 348:43-62.
6. Gotoh M, et al. Increased interleukin-1 beta production in the synovium of glenohumeral joints with anterior instability. J Orthop Res 1999; 17:392-97
7. Gotoh M, et al. Increased substance P in subacromial bursa and shoulder pain in rotator cuff diseases. J Orthop Res 1998; 16:618-21.
8. Hatse S, Princen K, Bridger G, et al. Chemokine receptor inhibition by AMD3100 is strictly confined to CXCR4. FEBS Lett 2002; 527:255-62.
9. Hirschmann M T, Zschäbitz A, Stofft E. Immunohistochemical Characterization of Human Synovial Bursa Cells by Light and Transmission Electron Microscopy. Where do These Cells Come From? Int J Morphol 2007; 1:5-14.
10. Hitchon C, Wong K, Ma G, et al. Hypoxia-induced production of stromal cell-derived factor 1 (CXCL12) and vascular endothelial growth factor by synovial fibroblasts. Arthritis Rheum 2002; 46:2587-97.
11. Ingegnoli F, Blades M, Manzo A, et al. [Role of cell migration in the pathogenesis of rheumatoid arthritis: in vivo studies in SCID mice transplanted with human synovial membrane]. Reumatismo 2002; 54:128-32.
12. Ishii H, Brunet J A, Welsh R P, et al. “Bursal reactions” in rotator cuff tearing, the impingement syndrome, and calcifying tendinitis. J Shoulder Elbow Surg 1997; 6:131-6.
13. Kanbe K, Takagishi K, Chen Q. Stimulation of matrix metalloprotease 3 release from human chondrocytes by the interaction of stromal cell-derived factor 1 and CXC chemokine receptor 4. Arthritis Rheum 2002; 46:130-7.
14. Kim Y S, Bigliani L U, Fujisawa M, et al. Stromal cell-derived factor 1 (SDF-1; CXCL12) is increased in subacromial bursitis and downregulated by steroid and non-steroidal anti-inflammatory agents. J Orthop Res 2006; 24:1756-64.
15. Nanki T, Hayashida K, El-Gabalawy H S, et al. Stromal cell-derived factor-1-CXC chemokine receptor 4 interactions play a central role in CD4+ T cell accumulation in rheumatoid arthritis synovium. J Immunol 2000; 165:6590-8.
16. Neer C S. Anterior acromioplasty for the chronic impingement syndrome in the shoulder. Bone Joint Surg Am 1972; 87:1399.
17. Proto A, Bigliani L U, Blaine T A. IL-1 induces expression of stromal derived factor-1 in subacromial bursal cells. Presented at the 6^{th} Combined Meeting of the Orthopaedic Research Societies; October, 2007.
18. Santavirta S, et. Al. Inflammation of the subacromial bursa in chronic shoulder pain. Arch Orthop Trauma Surg 1992; 111:336-40.
19. Singh S K, Morbach H, Nanki T, et al. Differential expression of chemokines in synovial cells exposed to different Borrelia burgdorferi isolates. Clin Exp Rheumatol 2005; 23:311-22.
20. Voloshin I, Gelinas J, Maloney M, et al. Proinflammatory cytokines and metalloproteases are expressed in the subacromial bursa in patients with rotator cuff disease. Arthroscopy 2005; 21:1076.
21. Blades, M. C., et al., Stromal cell-derived factor 1 (CXCL12) induces monocyte migration into human synovium transplanted onto SCID Mice. Arthritis Rheum, 2002. 46(3): p. 824-36.
22. Bradfield, P. F., et al., Rheumatoid fibroblast-like synoviocytes overexpress the chemokine stromal cell-derived factor 1 (CXCL12), which supports distinct patterns and rates of CD4+ and CD8+ T cell migration within synovial tissue. Arthritis Rheum, 2003. 48(9): p. 2472-82.
23. Garcia-Moruja, C., et al., Functional characterization of SDF-1 proximal promoter. J Mol Biol, 2005. 348(1): p. 43-62.
24. Hitchon, C., et al., Hypoxia-induced production of stromal cell-derived factor 1 (CXCL12) and vascular endothelial growth factor by synovial fibroblasts. Arthritis Rheum, 2002. 46(10): p. 2587-97.
25. Ingegnoli, F., et al., [Role of cell migration in the pathogenesis of rheumatoid arthritis: in vivo studies in SCID mice transplanted with human synovial membrane]. Reumatismo, 2002. 54(2): p. 128-32.
26. Kanbe, K., K. Takagishi, and Q. Chen, Stimulation of matrix metalloprotease 3 release from human chondrocytes by the interaction of stromal cell-derived factor 1 and CXC chemokine receptor 4. Arthritis Rheum, 2002. 46(1): p. 130-7.
27. Kanbe, K., et al., Synovectomy reduces stromal-cell-derived factor-1 (SDF-1) which is involved in the destruction of cartilage in osteoarthritis and rheumatoid arthritis. J Bone Joint Surg Br, 2004. 86(2): p. 296-300.
28. Nanki, T., et al., Stromal cell-derived factor-1-CXC chemokine receptor 4 interactions play a central role in CD4+ T cell accumulation in rheumatoid arthritis synovium. J Immunol, 2000. 165(11): p. 6590-8.
29. Singh, S. K., et al., Differential expression of chemokines in synovial cells exposed to different Borrelia burgdorferi isolates. Clin Exp Rheumatol, 2005. 23(3): p. 311-22.
30. Peng H, Erdmann N, Whitney N, Dou H, Gorantla S, Gendelman H E, Ghorpade A, Zheng J. HIV-1-infected and/or immune activated macrophages regulate astrocyte SDF-1 production through IL-1 beta. Glia 2006; 54(6):619-29.
31. García-Vicuña R, Gómez-Gaviro M V, Domínguez-Luis M J, Pec M K, González-Alvaro I, Alvaro-Gracia J M, Díaz-González F. CC and CXC chemokine receptors mediate migration, proliferation, and matrix metalloproteinase production by fibroblast-like synoviocytes from rheumatoid arthritis patients. Arthritis Rheum 2004; 50(12):3866-77.
32. Kaplan A P. Chemokines, chemokine receptors and allergy. Int Arch Allergy Immunol. 2001; 124(4):423-31.
33. Omoigui S, Irene S. Subcutaneous injection of anakinra in patients with shoulder pain due to rotator cuff tendonitis and subacromial bursitis. Pain Med. 2004; 5(2):229-30.
34. Singh J A, Christensen R, Wells G A, Suarez-Almazor M E, Buchbinder R, Lopez-Olivo M A, et al. Biologics for rheumatoid arthritis: an overview of Cochrane reviews. Cochrane Database of Systematic Reviews 2009, Issue 4. Art. No.: CD007848.
35. Gibbons L J, Hyrich K L. Biologic therapy for rheumatoid arthritis: clinical efficacy and predictors of response. BioDrugs. 2009; 23(2):111-24.
36. Caruz A, Samsom M, Alonso J M, Alcami J, Baleux F, Virelizier J L, Parmentier M, Arenzana-Seisdedos F. Genomic organization and promoter characterization of human CXCR4 gene. FEBS Lett. 1998; 426(2):271-8.
37. Mertens M, Singh J A. Anakinra for rheumatoid arthritis. Cochrane Database Syst Rev. 2009; (1):CD005121.
38. Kim Y S, Bigliani L U, Fujisawa M, Murakami K, Chang S S, Lee H J, Lee F Y, Blaine T A: Stromal Cell-Derived Factor 1 (Sdf-1; Cxcl12) Is Increased In Subacromial Bursitis And Downregulated By Steroid and Non-steroidal Anti-inflammatory Agents. J. ORTHOP. RES, 24(8): 1756-1764, 2006.

What is claimed is:

1. A method for treating or ameliorating a musculoskeletal disorder in a subject in need thereof, comprising administering to said subject a compound that inhibits or reduces binding or expression of Stromal Cell-Derived Factor-1α (SDF-1α) and CXCR4, thereby treating or ameliorating said musculoskeletal disorders.

2. The method of claim 1, wherein said compound is administered locally to a musculoskeletal tissue.

3. The method of claim 1, wherein said compound is a small molecule inhibitor.

4. The method of claim 3, wherein said small molecule inhibitor is a compound having the formula I: 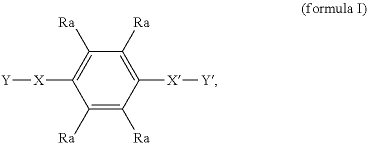
or a pharmaceutically acceptable salt, solvate, or prodrug thereof, where
X and X′ are independently selected from (a) a bond, (b) C₁-C₆ alkyl, (c) C₂-C₆ alkenyl, (d) C═NR¹, (e) CO, (f) C(O)NR¹, (g) NR¹, and (h) CHNNR¹;
wherein (b)-(c) is optionally substituted with one or more —N— or —NR¹—;
Y and Y′ are independently selected from (a) NR¹R¹, (b) C(═NR¹)NR¹R¹, (c) C₃-C₁₈ membered saturated, unsaturated, or aromatic carbocycle, and (d) C₃-C₁₈ membered saturated, unsaturated, or aromatic heterocycle containing one or more heteroatoms selected from nitrogen, oxygen, or sulfur;
wherein (c)-(d) is optionally substituted with one or more R² groups;
alternatively, Y and Y′ are NR¹R¹R¹;
Ra, at each occurrence, independently, is selected from (a) hydrogen, (b) C₁-C₆ alkyl, (c) C₁-C₆ alkoxy, and (d) halogen;
R¹, at each occurrence, independently, is selected from (a) hydrogen and (b) C₁-C₆ alkyl; and
R², at each occurrence, independently, is selected from (a) C₁-C₆ alkyl, (b) OH, (c) C₁-C₆ alkoxy, (d) C₃-C₁₄ membered saturated, unsaturated, or aromatic carbocycle, and (e) C₃-C₁₄ membered saturated, unsaturated, or aromatic heterocycle containing one or more heteroatoms selected from nitrogen, oxygen, or sulfur;
or a compound having the formula Ia: 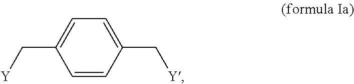
or a pharmaceutically acceptable salt, solvate, or prodrug thereof; wherein Y and Y′ are as defined above.

5. The method of claim 4, wherein said small molecule inhibitor is selected from the group consisting of 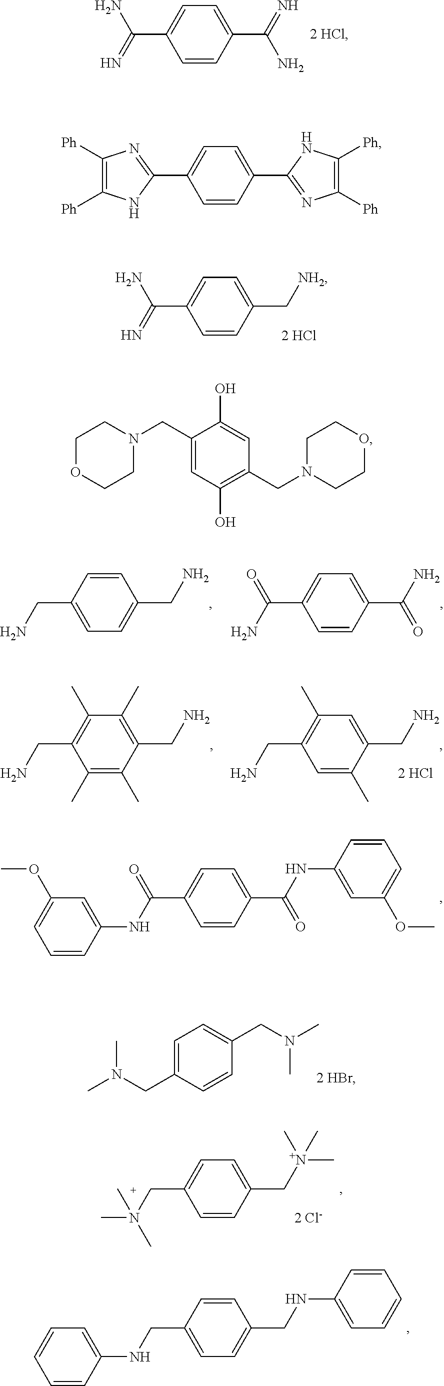 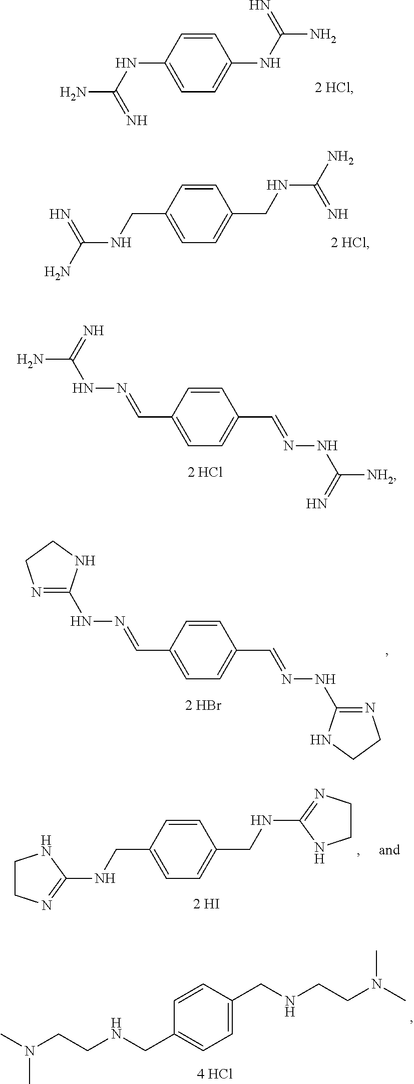
or a pharmaceutically acceptable salt, solvate, or prodrug thereof.

6. The method of claim 3, wherein said small molecule inhibitor is AMD 3100.

7. The method of claim 1, wherein said compound is a peptide inhibitor.

8. The method of claim 7, wherein said peptide inhibitor comprises a sequence of SEQ ID NO: 7, 8, 9, or 10.

9. The method of claim 7, wherein said peptide inhibitor is a T140 analog.

10. The method of claim 1, wherein said musculoskeletal disorder comprises bursitis.

11. The method of claim 1, wherein said musculoskeletal disorder comprises tendonitis.

12. The method of claim 1, wherein said musculoskeletal disorder is selected from the group consisting of rotator cuff tendonitis, achilles tendonitis ankle, patellar tendonitis, tennis elbow, trochanteric bursitis, epicondylitis olecranon bursitis, subacromial impingement syndrome, and subacromial inflammation.

13. The method of claim 2, wherein said musculoskeletal tissue is a bursa or tendon.

14. The method of claim 1, wherein said compound inhibits or binds to SDF-1α.

15. The method of claim 1, wherein said compound inhibits or binds to CXCR4.

16. The method of claim 1, wherein said CXCR4 comprises amino acids consisting of SEQ ID NO: 4.

17. The method of claim 1, wherein said CXCR4 comprises amino acids consisting of SEQ ID NO: 6.
